(19) United States
(12) Patent Application Publication
(10) Pub. No.: US 2003/0153729 A1
(43) Pub. Date: Aug. 14, 2003

(54) Enzyme/chemical reactor based protein processing method for proteomics analysis by mass spectrometry

(76) Inventors: Henry S. Duewel, Mississauga (CA); Theo Goh, Toronto (CA); Thierry LeBihan, Mississauga (CA)

Correspondence Address: ROPES & GRAY LLP, ONE INTERNATIONAL PLACE, BOSTON, MA, 02110-2624, US

(21) Appl. No.: 10/330,859

(22) Filed: Dec. 26, 2002

Related U.S. Application Data
(60) Provisional application No. 60/343,859, filed on Dec. 28, 2001.

(51) Int. Cl. C07K 17/00

(52) U.S. Cl. 530/344; 422/99; 530/413

(57) ABSTRACT

A method and tools for the biochemical processing of protein samples towards their analysis and identification by, for example, mass spectrometry (MS). The method involves the reversible (non-covalent) immobilization, purification and concentration of proteins onto a solid surface and subsequent solid-phase enzyme-catalyzed proteolysis of the proteins. The constituent peptides are recovered in near-quantitative yields in a format ideally suited for identification by routine MS technologies. The process is easily adapted such that additional chemical and/or enzymatic transformation(s) of the immobilized proteins or peptides can be performed to facilitate the acquisition of and increase the information content obtained from the MS analysis.

### REFERENCE TO RELATED APPLICATIONS

The present application claims priority to U.S. Provisional application No. 60/343,859, filed on Dec. 28, 2001, the entire content of which is incorporated by reference herein.

### FIELD OF THE INVENTION

The disclosed invention relates generally to the field of proteomics. More specifically the invention encompasses tools and procedures for enzymatic and/or chemical transformation of polypeptides. The transformed polypeptides serve as either analytes or products for a number of applications.

### BACKGROUND OF THE INVENTION

Accurate and rapid sequence analysis of proteins and polypeptides has become crucial in the fields of biology, biotechnology and proteomics. Despite completion of the mapping of the human genome, a possible half million human proteins encoded by some 30,000 genes must still be identified and characterized. The new discipline of proteomics aims to unravel biochemical information at the molecular level.

At present, only a limited number of techniques have been developed to address the growing need to quickly and accurately identify proteins through mass-accurate methods such as mass spectrometry. These current protein identification procedures and platforms involve the integration of three broad practices, including sample production or isolation, sample processing and sample analysis. The practices of sample production and analysis must be coupled by effective sample processing methods for optimal implementation of a platform. Once the protein sample is isolated, sample processing routinely includes protein purification, preconditioning (e.g. reduction and alkylation of cysteine residues) and concentration followed by degradation of the purified protein into constituent peptide fragments, either by chemical degradation or, more commonly, by enzymatic hydrolysis (e.g. by use of proteases). The peptides so produced are recovered and analyzed by mass spectrometry. This mass spectrometric data is then correlated with information contained in genomic and protein databases in order to identify the proteins in the original sample.

Conventional proteomic platforms rely heavily on either one- or two-dimensional gel electrophoresis for sample processing. Here, separation, purification and concentration of protein samples occur during electrophoresis. Following electrophoresis, separated proteins in the gel are visualized through differential staining and the protein-containing gel bands or spots of interest are excised. The protein-containing gel band or spot can then be treated to ‘in-gel’ chemical or enzymatic digestion, followed by extraction of the constituent peptides for later sequencing. Such gel-based processing method requires subjecting the gel band or spot to a series of drying and swelling procedures, in order to introduce the reagents for digestion and to extract the digested fragments. Alternatively, the protein can be transferred to a PVDF membrane for digestion or automated Edman degradation.

Significant problems exist with conventional gel-based processing platforms. Many proteins cannot enter the gel, or be separated in-gel, because of their physicochemical properties. In particular, hydrophobic proteins or proteins of high molecular mass are poorly processed by gel methods. Furthermore, detectability is limited to the sensitivity of the stain used for visualization. Proteins below this level, by definition, are precluded from subsequent analyses. Additionally, inherent limitations to the ‘in-gel’ chemical or enzymatic digestion of the proteins currently exist which lowers the overall sensitivity and performance of the method. These include the adverse effects of the gel matrix on the digest efficiency and the recovery of the constituent peptides.

Furthermore, gel-based processing platforms, by necessity, require numerous sample handling procedures and sample transfers. One implication of successive manipulations of the sample is loss of the protein prior to the digestion step, as well as loss of the constituent peptides prior to analyses. The losses at each step are multiplicative, and compromise the overall sensitivity of the gel-based platform. Another implication of successive manipulations of the sample, especially when the methods are performed manually, is the increased potential of sample contamination, such as from dust or keratin (a protein in human skin and hair).

An attempt has been made to automate electrophoretic separations of macromolecules and, in particular, to automate two-dimensional electrophoretic separations used in the analysis of proteins (Anderson et al., U.S. Pat. No. 6,245,206, 2001). Such two-dimensional procedures involve sequential separation of the proteins by isoelectric focusing (IEF) and sodium dodecylsulfate polyacrylamide gel electrophoresis (SDS-PAGE), and a method involving the use of robotics and computer software to automate the manipulation of two-dimensional gel media. However, such a system is complex, capital intensive, inflexible and costly.

As an alternative to gel-based sample processing methods, other proteomic platforms rely on solution-phase sample processing methods. Here, more conventional sample production approaches, such as sub-cellular fractionation, affinity chromatography, immunoprecipitation, and various other standard chromatography techniques (e.g. ion exchange, size-exclusion, or hydrophobic chromatography) are used to isolate the protein sample. Then, chemical or enzymatic digestion of the proteins into their constituent fragments is performed in solution, either directly or following the addition of other reagents, as required, or following additional sample processing manipulations, as required.

The effectiveness of conventional solution-phase processing methods depends upon a number of practical considerations. First, the chemical composition of the protein sample will be determined by the buffer components required for the solution-phase protein production method employed. Often, protein isolation requires the use of high concentrations of salts, detergents or other additives that ultimately contaminate and dilute the sample. The presence of such contaminating additives can interfere with the efficient digestion of the protein into its constituent fragments during sample processing. The chemical and/or enzymatic transformation of proteins often requires a specific set of solution conditions, such that the chemical environment of the protein sample (as determined through the application of conventional solution-phase sample processing) may result in the sample being unresponsive or incompatible to further chemical or enzymatic degradation. Furthermore, such contaminants may also interfere with the mass spectrometric analysis of the product peptides, greatly reducing the quality of the data obtained.

Additionally, conventional solution-phase purification approaches result in the protein sample being obtained at dilute concentrations (e.g. sub micromolar range) in relatively large volumes (Figeys, D. and Pinto, D. Electrophoresis (2001) 22, 208-216). This reduces the efficiency of the chemical or enzymatic digestion of the protein into its constituent fragments. For example, most commonly used proteases in proteomic platforms (e.g. trypsin) have Kₘ values in the low- to mid-millimolar range, and the digestion efficiency is significantly reduced as the concentration of protein is reduced below these levels. As such, the sample usually requires a pre-concentration step in order for the digestion reaction to proceed in a kinetically favourable manner. Pre-concentration methods such as protein precipitation, solution evaporation or dry-down and ultrafiltration concentration introduce additional sample handling steps and, therefore, as in the case of gel-based processing, are sources of sample loss. The losses at each step are multiplicative and compromise the overall sensitivity of the solution-phase processing platform. In addition, any preconcentration method will also result in the concentration of contaminants as well as the sample of interest; for reasons already discussed this is undesirable.

The field of proteomics generates very small quantities of sample indeed. For example, it may be desirable to analyze the proteome of a tissue from a single individual. In such cases it may only be possible to obtain milligram quantities of whole tissues, which will contain only microgram quantities of total protein. Current sample processing methodologies are not capable of handling such limited quantities of protein efficiently.

Mass spectrometry (MS) provides a means of determining the mass of polypeptide digest fragments. Under the influence of electric and/or magnetic fields, the peptide-derived ions follow trajectories depending on their individual mass (m) and charge (z). Many applications of mass spectrometric methods are well known in the art (Meth. Enzymol., 193); (McCloskey, ed.; Academic Press, NY 1990; McLaffery et al., Acc. Chem. Res. 27:297-386 (1994); Chait and Kent, Science 257:1885-1894 (1992); Siuzdal(, Proc. Natl. Acad. Sci., USA 91:11290-11297 (1994)). The spectrum of fragment masses can then be searched against a database to determine the sequence of the target protein (mass fingerprinting). In cases where mass fingerprinting cannot provide all the necessary data, “tag sequencing” (tandem MS/MS peptide sequencing) can be used (Gross, Trends in Anal. Chem. 17 (1998) 470).

A variety of mass spectrometry techniques are currently used in the sequencing of proteins, including Time of Flight (Tof), Q-trap, quadrupole-Tof (Qtof), ion-trap, MALDI Time of Flight (MALDI-Tof); Fourier-Transform mass spectrometry (FT-MS) and tof-tof. Ionization techniques which may be employed include Matrix Assisted Laser Desorption/Ionization (MALDI) (Hillenkamp, Anal. Chem. 60 (1988) 2299); and Electrospray Ionization (ESI) mass spectrometry.

The use of mass spectrometry for identifying proteins has increased the importance of sample processing in order to provide a sample ideally suited for use in MS techniques. Sample processing methods for gel-based or solution-phase methods is a ‘bottleneck’ to the performance limits of detection (LOD) of the mass spectrometer. Current mass spectrometry technology is able to analyze peptides at the attomolar level (10⁻¹⁸ mol); however, existing upstream sample handling processes, particularly in the case of gel-based or solution-phase methods, can at best process proteins obtainable in approximately femto- to lower pico-molar amounts. This is due to the fact that losses are significant with the conventional gel-based and solution-phase sample processing methods.

Hsieh discloses a technique for automation of multidimensional separations through columns of differing selectivities (Hsieh, Anal. Chem. 1996). First, the target protein is purified by immunoaffinity chromatography. The protein is then digested in an immobilized trypsin column, after which the tryptic digest is transferred to a perfusion dilute capture column where it is concentrated and desalted. Peptides eluted from the dilute capture column are analyzed by single-stage mass spectrometry (MS) or tandem MS/MS. Although this technique eliminates the amount of handling necessary for a particular sample, it requires relatively complicated multi-column chromatography.

Finally, Little et. al. (U.S. Pat. No. 6,322,970 B1, 2001) discloses a method of obtaining a target polypeptide, immobilizing the target polypeptide to a solid support (preferably with a linker which is acid cleavable, acid-labile, heat sensitive or photo-cleavable), treating the immobilized target polypeptide with an enzyme or chemical to generate a series of deleted fragments, conditioning the cleaved fragments, cleaving the linker to release the immobilized fragments and determining the mass of the released fragments. However, Little discloses the use of a covalent linker to the substrate, and does not teach pH-regulated immobilization of the polypeptides to an ionic substrate.

The above techniques suffer from a number of disadvantages. For example, in the case of solution-based methods such as LC and HPLC disclosed above, the protein substrates are subjected to enzymatic and chemical transformations in solution or are transiently passed over a solid surface bearing immobilized enzymes or chemical reagents, thus reducing the efficiency of LC or HPLC relative to a situation in which proteins are surface immobilized.

An extended discipline of proteomics has recently emerged, termed quantitative proteomics. The discipline makes use of mass spectrometry and the proven technique of stable isotope labeling [De Leenheer, A. P. and Thienpont, L. M. (1992) Mass Spectrom. Rev. 11, 249-307] as a means of protein quantification. The method involves the addition to the sample of a chemically identical form of the mass analyte (e.g. polypeptide fragment) that contains, or is labeled with, stable heavy isotopes (e.g. ²H vs. ¹H, ¹³C vs. ¹²C, ¹⁸O vs. ¹⁶O). In its simplest form, polypeptides derived from one sample (e.g. State A) are combined with polypeptides derived from a second sample (e.g. State B). The polypeptides from State A are processed such that they are labeled with the “light” (i.e. unlabeled) version of an elemental isotope. By contrast, the polypeptides from State B are processed such that they are labeled with the “heavy” version of the same element. If both samples from State A and State B contain the same polypeptides, but at different relative amounts, then all polypeptides in the combined mixture will exist as analyte pairs of identical sequence and chemical composition, but will differ in mass by an amount equal to the difference between the light and heavy isotope. Thus, the ratio between the mass intensities of the light- and heavy-labeled polypeptide pair, as determined by routine mass spectrometry analysis, will provide a measure of the relative abundance of the polypeptide in the original samples, namely State A and State B.

Several applications of stable heavy isotope labeling for quantitative proteomics using mass spectrometry are known in the art. These include the practice of metabolic labeling of proteins in vivo during protein synthesis for example, with ¹⁴N or ¹⁵N [Conrads, T. P., Alving, K., Veenstra, T. D., Belov, M. E., Anderson, G. A., Anderson, D. J., Lipton, M. S., Pasa-Tolic, L., Udseth, H. R., Chrisler, W. B., Thrall, B. D., and Smith, R. D. (2001) Anal. Chem. 73, 2132-2139] or with labeled amino acids such as leucine-¹H₁₀ or leucine-²H₁₀ [Martinovic, S., Veenstra, T. D., Anderson, G. A., Pasa-Tolic, L., and Smith, R. D. (2002) J. Mass Spectrom. 37, 99-107]. Other practices include the chemical labeling of proteins in vitro, which exploit the selective reactivity of an amino acid residue to derivatization with a light and heavy form of a chemical reagent or tag. For example, cysteine residues in proteins have been labeled using the light (¹H₈) and heavy (²H₈) forms of the ICAT reagent [Gygi, S. P., Rist, B., Gerber, S. A., Turecek, F., Gelb, M. H., and Aebersold, R. (1999) Nat. Biotechnol. 17, 994-999], or with the light (¹H₃) and heavy (²H₃) forms of acrylamide [Sechi, S. (2002) Rapid Commun. Mass Spectrom. 16, 1416-1424].

Another practice of stable isotope labeling involves the use of ¹⁸O-labeled water (H₂¹⁸O) in the course of the sample processing step of polypeptide digestion by proteases. All proteases commonly used in proteomic platforms (e.g. trypsin) catalyze hydrolysis of peptides bonds in which a water molecule from the bulk solvent reacts with a peptide carbonyl group, and the bond to the amino group of the next amino acid is cleaved [Kraut, J. (1977) Annu. Rev. Biochem. 46, 331-358]. By virtue of the catalytic mechanism, each proteolytic peptide generated (with exception of the C-terminal peptide of the protein) is expected to have at its carboxy terminus one oxygen atom originating from the original peptide carbonyl group and one oxygen atom incorporated from the bulk solvent. Thus, when ¹⁸O-enriched water is present in the bulk solvent the extent of ¹⁸O incorporation into each peptide will be a function of the relative H₂¹⁸O/H₂¹⁶O ratio present in the digestion buffer.

The practice of ¹⁸O-labeling is receiving increasing attention as a preferred method for heavy isotope labeling and several examples of its application have appeared [Stewart, I. I., Thomson, T. and Figeys, D. (2001) Rapid Commun. Mass Spectrom., 15, 2456-2465; Wang, Y. K., Ma, Z., Quinn, D. F. and Fu, E. W. (2001) Anal. Chem., 73, 3742-3750; Yao, X., Freas, A., Ramirez, J., Demirev, P. A. and Fenselau, C. (2001) Anal. Chem., 73, 2836-2842]. In general, the application of ¹⁸O-labeling involkes the use of solution phase sample processing although reports using gel-based sample processing in conjunction with ¹⁸O-labeling have also appeared [Kuster, B. and Mann, M. (1999) Anal. Chem. 71, 1431-1440; Kosalka, T., Takazawa, T. and Nakamura, T. (2000) Anal. Chem. 72, 1179-1185].

It is common practice and considered advantageous to use the highest enrichment of H₂¹⁸O as possible in order to achieve the highest degree of labeling of each proteolytic fragment for quantitative proteomics applications. A severe limitation to the ¹⁸O-labeling approach is the limited commercial availability and high cost of highly enriched (>95 atom % ¹⁸O) H₂¹⁸O [Ad Hoc Committee of the North American Society for the Study of Obesity (1999) in ‘Report on the Supply and Demand of ¹⁸O Enriched Water’, http://www.naaso.org/newsflash/oxygen.htm].

Current gel-based and solution-phase sample processing methods necessitate the use of relatively large volumes of H₂¹⁸O. In addition, especially in the case of solution-phase sample processing, the sample, which will be isolated in a medium containing only natural abundance water (i.e. H₂¹⁶O), must either be greatly diluted into H₂¹⁸O containing buffers or the sample must be dried-down and then reconstituted in a small volume of H₂¹⁸O-containing buffers in order to preserve a high enrichment percentage of H₂¹⁸O in the final sample to be processed. Often, these methods are not feasible or compatible with optimal sample processing required for effective enzymatic degradation of the proteins. For example, dilution of the sample will lead to lowering the concentration of the protein sample. Dry-down pre-concentration will also result in concentration of contaminants in the sample. The negative effect of the latter sample processing steps on the enzymatic digestion has been addressed earlier.

There is currently no single technique, or combination of techniques, which fulfils the exacting needs of proteomic analysis. Such a technique must address a number of requirements related to protein sample handling and processing, including:

a) reducing the number of sample transfers required, thus removing a significant source of sample loss,

b) the capability of being carried out on-line in an integrated format, and that can be significantly automated,

c) significantly increasing the amount of protein that is captured and analyzed to near quantitative yields, preferably without the need of gel-based separations,

d) efficiently processing the very small quantities of material which a proteomics experiment generates, without the need for multiple experiments or sample pooling,

e) consists of a single sample handling and loading step, and that does not require the transfer of protein samples onto and off of a gel or membrane, thus significantly reducing protein loss and dramatically reducing the labour required,

f) the capability that the sample volume of the final peptide solution should be significantly reduced, such that the entire sample is typically eluted in less than 10-20 μL of buffer,

g) increasing the efficiency of proteolytic digestion of the proteins into peptide analytes and decreasing auto-digestion of the proteolytic enzyme,

h) the capability which allows for surface immobilization of sample proteins, thus increasing the effective concentration of the protein reactant 2-5 orders of magnitude compared to the equivalent solution phase reaction and allowing implementation of the chemical or enzymatic transformations that would otherwise not be possible in an industrialized platform via the corresponding solution based methodology,

i) allows for transfer of a protein sample isolated in relatively large volumes of natural abundance water (H₂¹⁶O) into a medium containing highly enriched H₂¹⁸O (>95 atom % ¹⁸O) without sample loss and allowing enzymatic digestion of the protein in said medium with concomitant high efficiency labeling of the proteolytic fragments with ¹⁸O at the carboxy terminus,

j) the ability to achieve the capability as outlined in (i) while using only minimal amounts of H₂¹⁸O (<50 μL) per experiment, thus significantly reducing the cost associated with using highly enriched H₂¹⁸O as a reagent, and

k) the capability as outlined in (i) and (j) that also encompasses requirements as outlined in (a-h).

### SUMMARY OF THE INVENTION

An invention, encompassing a number of tools, devices and procedures, has been developed which facilitates and improves upon current methods of polypeptide sample processing and current methods for the enzymatic and/or chemical transformation of polypeptides. The transformed polypeptides are recovered in high yields, in a format ideally suited for routine mass spectrometry based analyses in applications directed towards protein identification and quantitation. The invention is easily adapted such that additional chemical and/or enzymatic transformation(s) of the immobilized polypeptides can be performed in order to facilitate the acquisition of, and increase the information content obtained from, the mass spectrometry analysis.

Disclosed is a method and apparatus for the processing of protein or peptide samples for their analysis and identification by known means, such as mass spectrometry (MS). The method involves the reversible (i.e. non-covalent) immobilization or capture and concentration of proteins onto a solid surface and subsequent solid-phase transformation (such as chemical modification and/or enzyme-catalyzed proteolysis) of the proteins. In a preferred embodiment, the constituent peptides are recovered in near-quantitative yields in a format ideally suited for identification by MS methodologies, including, but not limited to, various types of detection platforms (e.g. time-of-flight, ion trap, quadrupole and Fourier-transform) and ionization source-interfaces (e.g. MALDI, electrospray, nano-electrospray, and liquid-chromatography-MS). Moreover, any other analytical device useful for the detection or identification of proteins or peptides, currently used or hereinafter invented, can be used. The process is easily adapted such that additional chemical and/or enzymatic transformation(s) of the immobilized proteins or peptides can be performed.

Also disclosed is a method for the rapid and reliable sequencing and analysis of proteins, utilizing mass spectrometry to identify proteolytic fragments to determine polypeptide sequences. In one embodiment, the method includes the reversible immobilization or ‘capture’ of proteins or peptides onto a solid support. In a preferred embodiment, such reversible immobilization may be by pH- or ionic strength-dependent immobilization of proteins to a strong anionic surface, such as a cationic exchange resin. More generally, such reversible immobilization also contemplates use of cationic substrates, ion exchange resins, as well as mixed bed resins, hydrophobic resins and affinity columns.

The method includes a next step in which the reversibly immobilized proteins are subjected to solid phase-based chemical or enzymatic modification of the said proteins or peptides. For example, the proteins, while remaining immobilized on the surface, are digested with a proteolytic enzyme into their constituent peptides. In a particular embodiment, trypsin is used to digest the immobilized protein. In another example, cysteine residues are chemically reduced with dithiothreitol (DTT) and allylated with iodoacetamide (IA).

The method also includes the further step in which peptide fragments produced from the proteolytic digestion are desorbed from the solid surface through a change in pH and/or cation or anion concentration and are recovered in an exceptionally small volume. The concentrated peptide solution requires no further purification and can be directly analyzed by MS techniques, including, but not limited to, various types of detection platforms (e.g. time-of-flight, ion trap, quadrupole and Fourier-transform) and ionization source-interfaces (e.g. MALDI, electrospray, nano-electrospray, and liquid-chromatography-MS). Moreover, any other analytical device useful for the detection or identification of proteins or peptides, currently used or hereinafter invented, can be used. The identity of the proteins can be obtained from database queries of MS derived data.

Also disclosed is a method that involves protein/peptide concentration and immobilization through ionic bonding of the proteins/peptides with the anionic surface through a cation exchange mechanism, and the subsequent chemical or enzymatic transformation of the immobilized protein/peptide in a highly efficient manner. In one particular embodiment, immobilization of the proteins/peptides is achieved when the proteins/peptides are applied to the surface using a low ionic strength (μ≦100 mM) buffer at pH 1-3. Under these conditions, all natural proteins/peptides will bear at least one cationic group and the net charge of the protein/peptide is greater than zero. For these reasons, the cationic protein/peptide moiety will exchange with a cationic counter-ion on the solid-surface and the protein/peptide becomes ionically bound to the surface immobilizing the protein/peptide to the surface. One advantage of this method is that the total amount of protein/peptide that can be immobilized onto the surface is dependent only upon the binding capacity and amount of solid-surface employed, and not on the initial concentration of the protein/peptide in the binding buffer. Therefore, proteins/peptides present in small to large volumes can be concentrated and immobilized onto a given amount of solid-surface.

Another advantage of the method is the increased efficiency of proteolytic digestion of the proteins into peptide analytes. Protein samples are denatured during the immobilization process and exist in extended conformations on the surface. This presents the proteolytic enzyme with a dramatically improved digestion target, resulting in improved peptide production. For example, digestion of the immobilized peptide or protein may give rise to fragments not found by solution digestion (due to limitations inherent in the kinetics of solution chemistry). Such information provides valuable sequencing information.

Another advantage is that the auto-digestion of the proteolytic enzyme, which can complicate MS analysis, is dramatically reduced. The described experimental protocol results in the observance of practically no peptide fragments from the endoprotease itself. Auto-digestion of the protease is a common problem in gel-based and solution-phase processing methods and reduces the quality of the MS analysis.

Yet another advantage is that surface immobilization of the proteins/peptides can, under practical conditions, increase the effective concentration of the protein/peptide reactants 2-5 orders of magnitude compared to the equivalent solution-phase reaction, thus allowing implementation of chemical or enzymatic transformations that would otherwise not be possible in an industrial platform which utilized solution- or gel-based methodologies.

A further advantage is that the disclosed method eliminates the performance ‘bottleneck’ that results from gel- or solution-based methods. Current mass spectrometry technology is able to analyze peptides at the attomole level (10⁻¹⁸ mol); however, existing upstream sample handling processes, particularly in the case of gel-based or solution phase methods, can at best process proteins obtainable in approximately femto- to lower pico-molar amounts.

In particular embodiments of the method, protein immobilization is followed by one or more wash steps. The wash steps are selected to preserve the ionic bonding (immobilization) of the proteins/peptides to the surface. They also allow for the removal of contaminating species, necessary for efficient enzymatic or chemical transformation of the immobilized proteins/peptides. Such contaminating species may also compromise subsequent MS performance.

The immobilized proteins/peptides can be subjected to essentially an unlimited number of transformations based on chemical and enzymatic reactions. In a preferred embodiment, the immobilized protein/peptides are digested with trypsin in an ammonium bicarbonate buffer (to maintain trypsin activity). Other chemical and enzymatic agents may be used to transform the reversibly immobilized proteins, including other endoproteases (e.g. chymotrypsin), exoproteases, kinases, methylases, glycosidases and phosphatases.

Also disclosed is a device that allows for the streamlined sample handling and processing method as disclosed herein, integrating protein immobilization and concentration, enzymatic or chemical transformation of the immobilized protein, and wash steps in a single column. In a particular embodiment the disclosed apparatus is a device that allows for on-line integration with standard MS devices, including MALDI, MALDI-Tof, Fourier transform MS, quadrupole MS, and ESI MS, or other techniques currently in use or hereinafter invented. In another embodiment, the disclosed device is a disposable high-throughput device.

In another embodiment, the disclosed device allows for sample volume of the final peptide solution to be significantly reduced, such that the entire sample is eluted in approximately 10-20 μL of buffer, dramatically increasing the concentration of the final peptide analytes and decreasing the need for further concentration. This greatly simplifies sample introduction to a mass spectrometer, for example.

Also disclosed is a device constructed from a fused silica column which houses a solid support (for example, a resin), a flow inlet and a flow outlet, wherein said solid support allows for reversible immobilization of a peptide or protein sample, enzymatic or chemical transformation of the sample into peptide fragments (or modified peptide fragments) while reversibly immobilized, and desorption of the peptide fragments from the solid support for collection and identification using any number of techniques, including mass spectrometry and Edman degradation, or other techniques currently known or hereafter invented.

Also disclosed is a device constructed from a capillary column that includes a strong cation exchange resin, a flow inlet and a flow outlet, such that the strong cation exchange resin in the column allows for reversible immobilization of a protein sample loaded into the column, along with a low pH buffer to lower the pH of the protein sample. The device allows for proteolytic digestion of the protein sample into peptide fragments while the fragments are reversibly immobilized, and desorption of the resulting peptide fragments from the solid substrate for collection and identification using mass spectrometry techniques. In particular embodiments, the device may be attached directly to a mass spectrometer for online collection and analysis of the protein fragments using standard mass spectrometry techniques.

Also disclosed is a method of constructing a device for the processing of peptide samples for analysis and identification by mass spectrometry, such that a column tube is packed with a solid substrate that allows for reversible immobilization of the protein. In a preferred embodiment, the solid substrate is a strong cation exchange resin. The column tube includes a flow inlet, and a flow outlet, such that when the protein sample is loaded into the column, the protein sample binds reversibly to the solid substrate. In particular embodiments, the protein sample is loaded into the device with a low-pH buffer, preferably in the range of pH 1-3. The device is constructed such that after proteolytic digestion of the peptide sample into peptide fragments while reversibly immobilized, the immobilized peptide fragments can be alternately washed using mobile wash steps, and subsequently desorbed and eluted from the solid substrate after treatment with a high pH buffer or high salt concentration. In particular embodiments, the device can be attached directly to a mass spectrometer for online collection and analysis of the protein fragments using standard mass spectrometry techniques.

### REFERENCE TO THE DRAWINGS

FIG. 1. Fabrication and components of a device incorporating the enzyme/chemical reactor using columns prepared with commercially available fused silica tubing fittings.

FIG. 2. Fabrication and components of a device incorporating the enzyme/chemical reactor using columns prepared with embedded strong cation exchange (SCX) resin/sol-gel frits.

FIG. 3. Schematic of the pressure vessel solvent delivery system for the method of the present disclosure.

FIG. 4. Schematic of the different modes of operation of the pressure vessel solvent delivery system showing operation of a 2-unit system.

FIG. 5. A schematic showing a 6-unit solvent delivery system, each unit of which may be operated independently.

FIG. 6. A silver-stained SDS-PAGE gel indicating that all proteins in the sample loaded onto the reactor became immobilized, and remained immobilized during subsequent wash steps. Lane (1) starting material (10 pmol/protein); (2) solution collected during the sample load step; (3) First wash fraction; and (4) Second wash fraction.

FIG. 7. Mass spectrum (MS) and tandem mass spectrum (MS/MS) for (1) Phosphorylase B peptide TNFDAFPDK; (2) Phosphorylase B peptide HLQIIYEINQR;

FIG. 8. Mass spectrum (MS) and tandem mass spectrum (MS/MS) for (1) BSA peptide LVNELTEFAK; (2) Carbonic anhydrase peptide VLDALDSIK.

FIG. 9. Coomassie-stained SDS-PAGE gel of human SKB1 bait protein and its interacting proteins as eluted from the antibody resin using 50 mM phosphoric acid as a method to characterize the immunopurified complex. Another portion of the eluent was subjected to trypsin digestion using the protocols described under Section C (Basic Protocol) and mass spectrometry. Shown in the Table are the proteins identified from the database search of the mass spectrometry-derived data.

FIG. 10. MALDI-TOF mass spectrum of the mixture of peptides 1, 2 and 3 after reduction and alkylation with iodoacetamide, following the instructions described under Section C (Alternate B Protocol). The graph indicates the relative proportions of the disulfide bonded, singly- and doubly-alkylated forms of peptides with 1, 2 and 3. Note that peptide 3 contains only 1 cysteine residue and thus, can only be singly-alkylated.

FIG. 11. MALDI-TOF mass spectra of peptide mass fingerprints from the control sample, which was processed by only enzymatic digestion, and the experimental sample, which was processed by chemical reduction, alkylation with iodoacetamide and enzymatic digestion with trypsin. (A) Bovine Serum Albumin peptides RPCFSALTPDETYVPK from the control sample and RPCFSALTPDETYVPK+1 iodoacetamide from the experimental sample; (B) Bovine Serum Albumin peptides LKPDPNTLCDEFKADEK from the control sample and LKPDPNTLCDEFKADEK+1 iodoacetamide from the experimental sample; (C) Bovine Serum Albumin peptides SLHTLFGDELCK from the control sample and SLHTLFGDELCK+1 iodoacetamide from the experimental sample.

### DETAILED DESCRIPTION OF THE INVENTION

Definitions

For convenience, certain terms employed in the specification, examples, and appended claims are collected here.

“Binding,” “bind” or “bound”, used interchangeably with “immobilizing”, “immobilize” or “immobilized”, refers to an association, which may be a stable association between two molecules, e.g., between a modified protein ligand an affinity capture reagent, due to, for example, electrostatic, hydrophobic, ionic and/or hydrogen-bond interactions under physiological conditions.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “Interacting Protein” is meant to include polypeptides that interact either directly or indirectly with a protein of interest (the “bait” protein). Direct interaction means that the proteins may be isolated by virtue of their ability to bind to each other (e.g. by coimmunoprecipitation or other means). Indirect interaction refers to proteins which require another molecule in order to bind to each other. Alternatively, indirect interaction may refer to proteins which never directly bind to one another, but interact via an intermediary.

The term “isolated”, as used herein with reference to the subject proteins and protein complexes, refers to a preparation of protein or protein complex that is essentially free from contaminating proteins that normally would be present in association with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

“Analyzing a protein by mass spectrometry” or similar wording refers to using mass spectrometry to generate information which may be used to identify or aid in identifying a protein. Such information includes, for example, the mass or molecular weight of a protein, the amino acid sequence of a protein or protein fragment, a peptide map of a protein, and the purity or quantity of a protein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either protein in its native state (e.g. as a part of a cell), or as part of a cell lysate, or that have been separated into components (e.g., in an acrylamide gel) but not obtained either as pure (e.g. lacking contaminating proteins) substances or solutions. The term isolated as used herein also refers to a component protein that is substantially free of cellular material or culture medium when produced by recombinant DNA techniques, or chemical precursors or other chemicals when chemically synthesized.

“Sample” as used herein generally refers to a type of source or a state of a source, for example, a given cell type or tissue. The state of a source may be modified by certain treatments, such as by contacting the source with a chemical compound, before the source is used in the methods of the invention. It should be noted that protein interaction network data based on “a sample” does not necessarily comprise results obtained from a single experiment. Rather, to completely determine a protein interaction network, multiple experiments are often needed, and the combined results of which are used to construct the protein interaction network data for that particular sample.

“Solid support” or “immobilization surface,” used interchangeably, refers to a material which is an insoluble matrix, and may (optionally) have a rigid or semi-rigid surface, and which has the capability to bind proteins or peptides. Such materials may take the form of small beads, pellets, disks, chips, dishes, multi-well plates, wafers or the like, although other forms may be used. In some embodiments, at least one surface of the substrate will be substantially flat.

“Transforming”, as used in “transforming protein” or the like, means change the original form of an intact protein (or fragment thereof) by one or more of: enzymatic or chemical digestion, modification (either on side chain or amino acid backbone, such as alkylation, etc.), reduction, oxidation, isotope labeling, covalently linking to a moiety, etc., or combination thereof (also called “serial transformation”).

The terms “reactor”, “device” and “apparatus” are somewhat interchangeable, and refer to various aspects of the invention disclosed herein. The reactor is the basic unit, comprising the solid support. The device comprises the combination of the reactor and components which allow it to be connected, for example, to a solvent delivery system. The apparatus is the combination of the device and a solvent delivery system. Optionally, the apparatus is further connected to an analytical device such as a mass spectrometer.

Overview

Disclosed is a method and device for allowing the purification, concentration and reversible immobilization of a protein in a nascent state onto a solid support, with the removal of any residual solvent and solute contaminants. The solid support may be a resin such as an ion-exchange resin, a hydrophobic support or an affinity support, for example. Ion exchange resins, including cationic, anionic and mixed bed resins may be used. In a preferred embodiment a cation exchange resin is employed. The immobilization surface may be fabricated in the form of a column (such as a fused-silica column whose internal wall forms the immobilization surface), a surface such as a glass chip or well, or a resin-packed pipette tip, for example. After immobilization and concentration of the protein, weakly cationic, neutral and anionic contaminating species are removed from the surface using appropriate mobile phase wash steps. The mobile phase wash steps are selected to preserve the immobilization of the proteins to the surface. The mobile phase wash steps also allow for the removal of contaminating species, necessary for subsequent enzymatic or chemical transformation of the immobilized proteins and to aid in resolution for sensitive MS detection of peptide fragments.

Following the above steps the immobilized protein is subjected to one or more enzymatic or chemical treatments. In a preferred embodiment, all solvent is removed from the immobilization surface prior to such treatment. This may be accomplished, for example, with pressurized gas (e.g. nitrogen or argon; which, opportunistically, provide an inert atmosphere) essentially dehydrating the surface of all solution. A key advantage of such a dehydration step is that the solution containing the enzymatic/chemical reagents can itself be used to rehydrate (or saturate) the immobilization surface, filling only the interstitial volume thereof (i.e. in a column format this is equivalent to adding only enough solution to fill the column void volume). For this reason, very small volumes of solution (˜50 nL) and amounts of reagents (˜pmol) are required. By such treatment, proteins remain either ionically bonded to the surface (in flow-through mode) or enter the mobile phase but remain within the surface bed but are not lost. This leads to a dramatic increase in the effective concentration of the reacting protein substrate and enzyme or chemical reactants.

Note that the dehydration/rehydration steps need not follow one another immediately. That is, after dehydration the immobilized proteins/peptides may be stored (under appropriate conditions) and processed at some later time. This advantage has obvious implications in automation, for example; a number of samples can be immobilized, dehydrated, and then transformed at a later stage of an automated process.

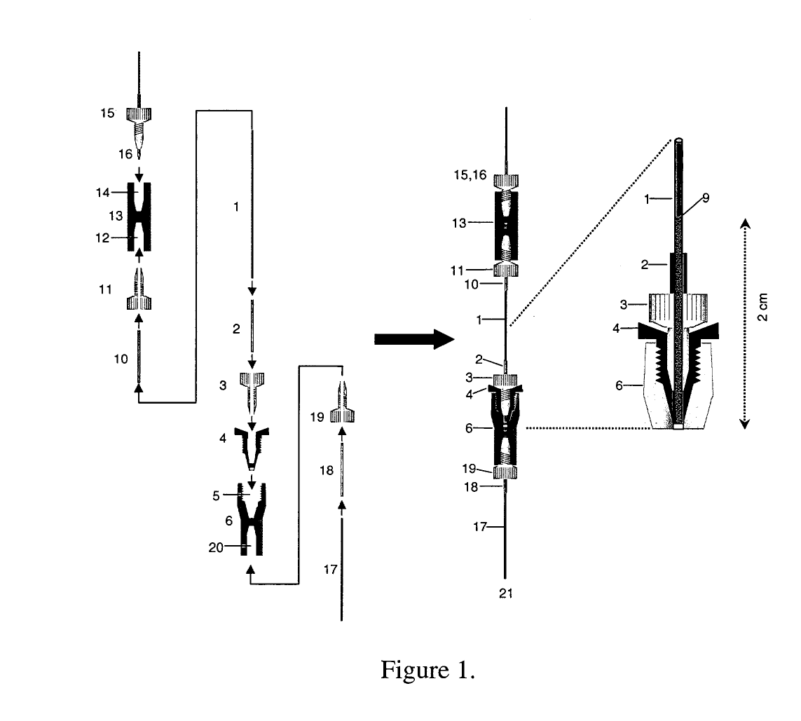

As described herein, two column formats for the reactor device are used. In one embodiment, columns are prepared using commercially available fused silica tubing fittings. The filter end fitting (4; FIG. 1) forms a physical plug or frit at the base of the column to constrain the SCX resin within the column. By necessity, this type of fitting requires the use and assembly of additional components to the column (2-4, 6, 17-19; FIG. 1) in order to form the flow outlet (21; FIG. 1).

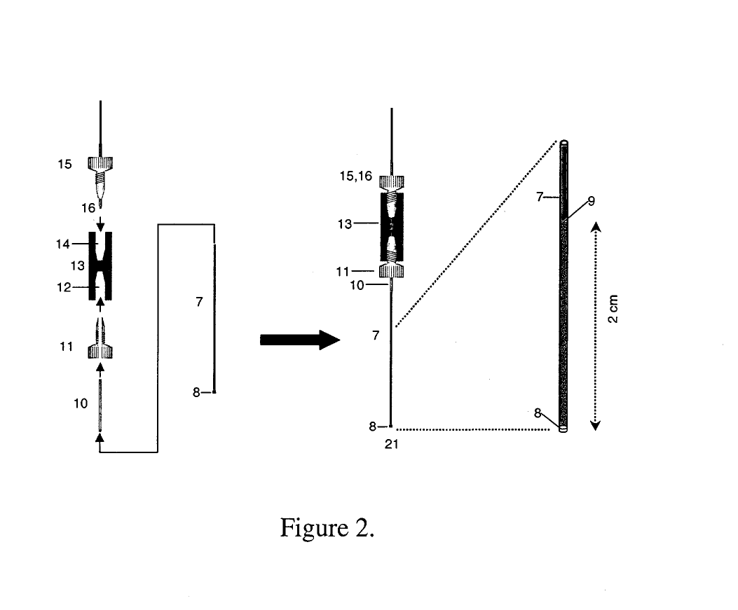

In the second embodiment, the reactor device is assembled using columns prepared with embedded SCX resin/sol-gel frits using a proprietary method. In this format, the physical plug or frit at the base of the column is physically integrated into the column, and thus, the column and the flow outlet of the column is formed from a single integrated component (7, 8, 21; FIG. 2). In this format, all the additional fittings required to form the column and the flow outlet (2-4, 6, 17-19; FIG. 1) for columns prepared using commercial fittings are not needed, which reduces the time required to assemble the device.

The first column format is described as an ‘easy to make’ alternative to those users who are not skilled in the art of sol-gel chemistry and the preparation of sol-gel embedded frits, since the components to fabricate the first format of the device are commercially available and no special skills are required. The second format of the column is described for users that have the capacity to become skilled in the art of sol-gel chemistry and the preparation of sol-gel embedded frits. The second format has some advantages over the first; these include the ability to prepare columns in large numbers in both a cost- and labour-effective manner. Additionally, one of the steps performed in routine operation of the reactor device, namely the rehydration step is performed more straightforwardly with columns prepared with embedded frits.

A preferred embodiment of the invention uses a device that requires a solvent delivery system capable of operating in the micro-flow realm for optimal operation. More specifically, a solvent delivery system is needed that can efficiently generate solvent flow rates at approximately 5 μL/min and, furthermore, generate said flow rates at a low operating pressure (<150 psi). Several micro-flow capillary HPLC instruments are commercially available that are capable of micro-flow solvent delivery. However, these instruments are extremely capital intensive. Furthermore, commercial instruments have basal operating pressures that typically exceed 500 psi and require relatively large volumes of solvents to completely purge and fill the internal fluid components (e.g. pumps, connective tubing).

In a preferred embodiment of the invention, the solvent delivery system must also be able to promote the complete transfer of a small volume of solvent (in cases, <5 μL) from a solvent vial to and then subsequently through the device. In addition, the solvent delivery system must also be able to operate in a stop-flow mode and further, be able to purge the device of all solvent preferably using an inert gas source. At present, commercial micro-flow HPLC instruments are incapable of meeting these requirements.

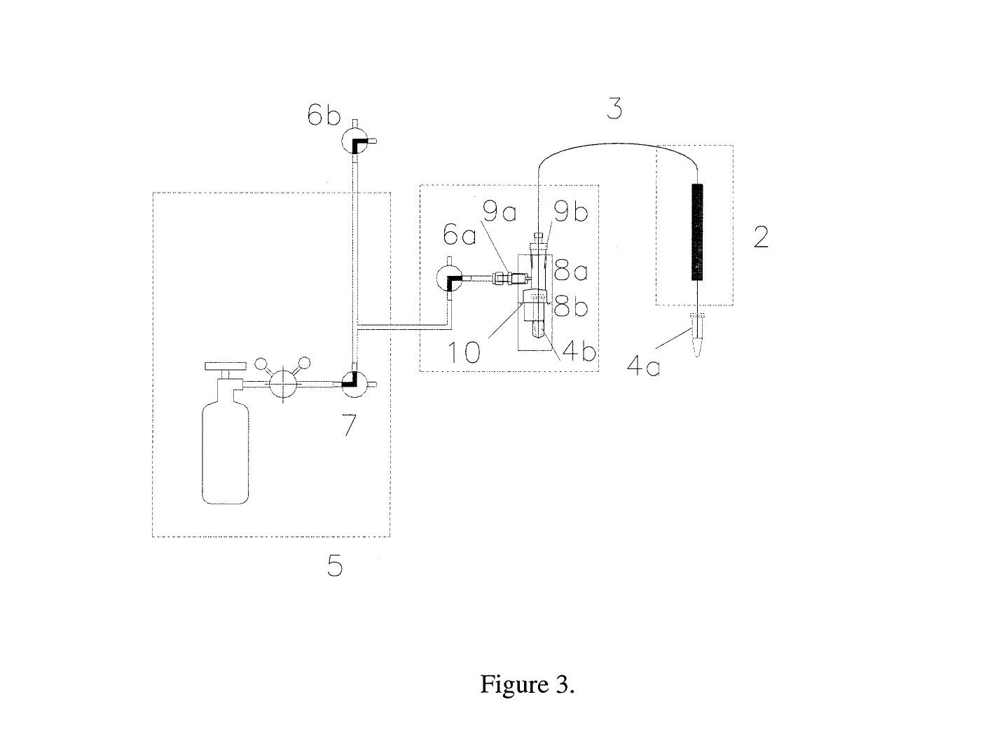

The solvent delivery system designed in-house is described herein. As illustrated in FIG. 3, a pressure vessel unit is constructed from two separate blocs of material (e.g. stainless steel or polymer). The upper bloc (8a) and the lower bloc (8b) are separable, and can be maintained together by constraining with screws, clamps or other mechanisms, thus forming a single unit. The O-ring (10) ensures that the unit is gas-tight. The lower bloc contains a cavity (or multiple cavities) of specific dimensions adapted for placement of a vial or tube. The upper bloc contains a cavity of similar dimensions, and is connected to a pressurized gas source via inlet connector (9a). One end of a fused silica tubing (3; usually 50 μm inner diameter by 360 μm outer diameter and 10-15 cm in length) is inserted exactly to the bottom of the vial (4b). The other end of the fused silica tubing is passed through a connector (9b), which holds it in place and forms a gas-tight seal. This end of the tubing forms the flow outlet of the pressure vessel solvent delivery system. This end of the tubing is inserted into a 1.5 cm length of capillary sleeve such that the ends of the tubing and the sleeve are flush. The sleeve-covered tubing is inserted into a fingertight fitting (15; FIGS. 1, 2) such that the end of the flush sleeve-covered tubing protrudes through the fingertight fitting by approximately 1 mm. The fingertight fitting is then screwed into the opening (14; FIGS. 1, 2) of the ZDV (zero dead volume) union (13; FIGS. 1, 2) of the flow inlet to the reactor.

In operation, a vial is filled with a desired volume of solvent and placed into the cavity of the lower bloc. The upper bloc and affixed tubing is aligned and brought together with the lower bloc and the blocs constrained together. The pressurized gas source is then opened. At sufficient pressure, the solvent in the vial is forced through the fused silica tubing towards the flow outlet of the pressure vessel. As the flow outlet of the pressure vessel is connected with the flow inlet of the device (2, FIG. 3), the solvent continues to flow through the reactor column, through the packed SCX resin and, finally, the solvent exits through the flow outlet of the reactor column into receptacle 4a. The operating pressure of the pressurized gas source controls the solvent flow rate. An operating pressure of approximately 100 psi results in a flow rate of approximately 5 μL/min with reactors fabricated with columns made with 200 μm inner diameter by 360 μm outer diameter fused silica tubing. An operating pressure of approximately 50 psi produces a flow rate of approximately 5 μL/min with reactors fabricated with columns made with 320 μm inner diameter by 425 μm outer diameter fused silica tubing.

The unit may be operated in semi-automatic mode by employing automatic actuated valves (6a and 6b, and 7), and feedback (i.e. automatic purging) can be ensured either by a micro-flow sensor after reactor (2), or by a micro switch.

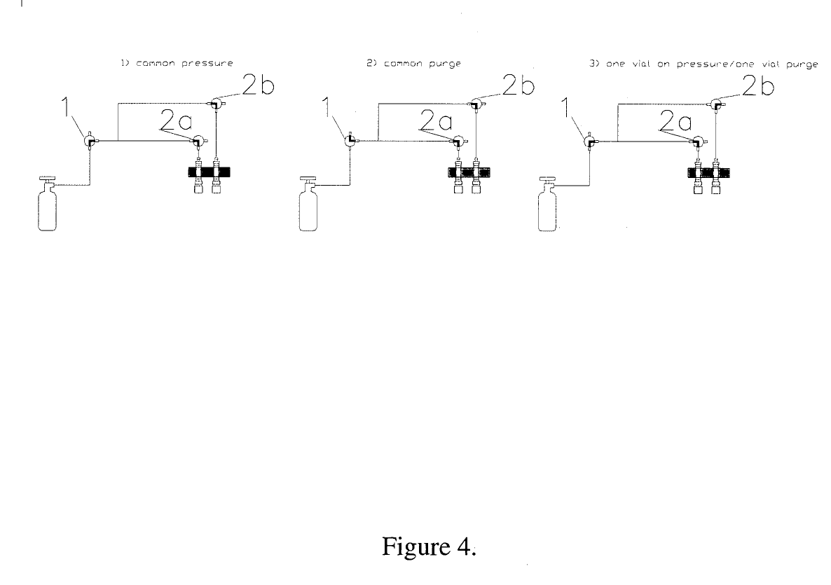

Multiple units may be combined, preferably in a suitable framework structure or manifold, to permit the simultaneous operation of multiple reactor devices. FIG. 4 illustrates the different modes of operation of such a device. For clarity, only 2 units are shown, but the number of units which may be so employed is not limited.

The 2 different valve positions are:

For valve 1: pressurized line OR purge line

For valve 2: connected gas line unit OR purge unit

In mode 1, valve 1 is in pressurized line mode AND valves 2 are in connected gas line unit mode, allowing the entire unit to be pressurized at the same time.

In mode 2, Valve 1 is in purge mode AND valves 2 are in connected gas line unit mode, thus allowing purging of the entire unit at the same time (by just using valve 1).

In mode 3, Valve 1 is in pressurized line mode, valve 2a is in connected gas line unit mode AND valve 2b is in purge unit mode. Thus, the pressure is maintained in unit 1 (liquid flow) and not in unit 2 (stop liquid flow).

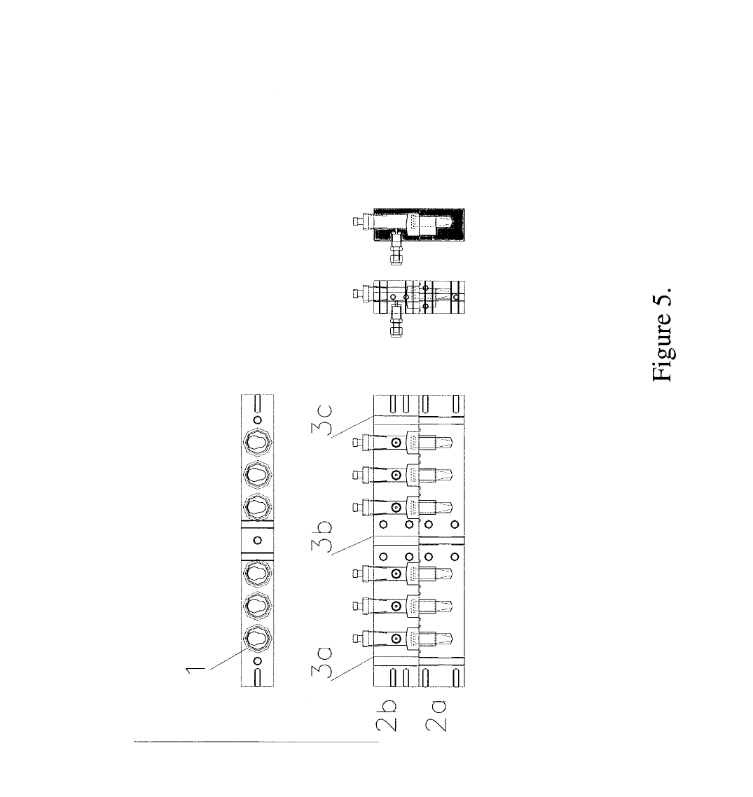

In a further refinement, illustrated in FIG. 5, each unit of a multi-unit device may be operated independently of all other units. Pressure independence between units is ensured by O-ring (1).

As described herein, two column formats for the reactor device are used. In one embodiment, columns are prepared using commercially available fused silica tubing fittings. The filter end fitting (4; FIG. 1) forms a physical plug or frit at the base of the column to constrain the SCX resin within the column. By necessity, this type of fitting requires the use and assembly of additional components to the column (2-4, 6, 17-19; FIG. 1) in order to form the flow outlet (21; FIG. 1).

Materials

The solid phase surface can be presented in the form of a cation exchange resin (beads) or as a cationic functionality bonded to the surface of chip (e.g. metal, glass) or polymeric membrane. The solid surface can be used in a batch format or can be housed in several formats including packed column and pipette tip. A preferred embodiment uses a strong cation exchange resin housed in a capillary illustrated in FIGS. 1 and 2 and described in section A, below. Protein samples can be present in aqueous or aqueous miscible organic solvents and the overall process can be performed manually or through automation. The protein/peptide can be either purified preparations or more complex samples derived directly from biological systems/organisms. Use of standard proteins and immunopurified protein complexes isolated from human cells is demonstrated at Examples 1 and 2, respectively.

Enzymatic and Chemical Transformations

In principle, the immobilized protein/peptide can be subjected to an unlimited number of transformations based on chemical and enzymatic reactions. For enzymatic reactions, a reaction solution containing the enzyme and any required co-factors is applied to the solid surface containing the immobilized protein/peptide, either in a flow-through manner or by allowing the enzyme solution to rehydrate the solid support. The enzyme solution is prepared and applied to the immobilized protein substrate using the optimal buffer conditions for the desired enzymatic reaction. The solid-phase enzymatic reaction is permitted to proceed for a certain time, after which the protein/peptide enzymatic products are displaced from the surface using 2-5 bed volumes of eluting buffer (for example, when using a cation exchange resin, a buffer having a high cation concentration and/or pH>˜8 is employed). The stringency of the eluting buffer can be adjusted to offer selective eluting capabilities.

The basic scope of the method can be extended to chemical reactions by simply applying a solution containing the desired chemical(s) to the immobilized protein/peptide surface. Exemplary procedures used for enzymatic reactions can be similarly applied to chemical transformations.

Nature of Enzymatic and Chemical Transformations

The method is applicable to a wide range of enzymatic reactions, and is restricted only by the availability of the desired enzyme and required cofactors. Although any enzymatic reaction involving the immobilized protein/peptide substrate can be performed using the described method, preferred embodiments focus primarily on the application of endoproteases (e.g. trypsin, chymotrypsin), kinases, glycosidases and phosphatases. Experimental results have been gathered for the proteolytic enzyme trypsin, as set forth in Examples 1, 2, 4 and 5. For a particular application in the case of endoprotease reactions, the reaction solution is prepared using heavy water (¹⁸O), resulting in the incorporation of the ¹⁸O label into the proteolytic peptides, as set forth in Example 5.

In the case of chemical reactions relevant to the invention, any type of solution phase protein chemistry (aqueous, organic or combination) can be used in the process including, but not limited to:

1. DTT and TCEP reduction of disulfides

2. sulfhydryl alkylation with any N-alkylated iodoacetamide derivative (e.g. iodoacetamide, ICAT, PEO Iodoacetyl Biotin)

3. carbodiimide-mediated activation or crosslinking

4. esterification of carboxylic acid groups

5. DANSYL chloride modification of the side chain of Lysine residues and N-terminal amino groups.

Serial Transformations

The method can be extended to allow several reactions to be performed on the same immobilized protein/peptide sample. Under carefully controlled wash conditions the first reaction solution (either chemical or enzymatic) can be removed from the surface while maintaining the immobilization of the protein/peptide to the surface. The resin can be washed and dehydrated as described previously. Then a second reaction solution (either chemical or enzymatic) can be applied to the surface-immobilized protein/peptide. The process can be repeated permitting several serial chemical or enzymatic reactions to be performed on the protein/peptide. An example of this principle has been achieved for serial reactions involving DTT reduction, sulfhydryl alkylation with iodoacetamide and digestion with trypsin (Example 4).

Mass Spectrometry Detection

The invention is applicable to several types of mass spectrometry detection platforms (e.g. time-of-flight, ion trap, quadrupole, and Fourier-transform) and ionization source-interfaces (e.g. MALDI, electrospray, nano-electrospray, and liquid-chromatography-MS). Although the method described is based on the use of mass spectrometry for protein analysis and identification, any other analytical device useful for the detection or identification of proteins/peptides can be used, either currently used or hereinafter invented. This could include fluorescence detection methods, UV spectroscopy, and radioactive isotope labeling.

A. Reactor Fabrication and Components (Refer to FIG. 1)

The production of a preferred device includes fabrication of the individual hardware components and processes to integrate these into the final working apparatus, as detailed below for a column reactor. These include a pre-conditioned SCX resin substrate; a fused silica column which houses the SCX resin (two formats are described); a process to pack the column with the pre-conditioned SCX resin; a flow inlet and flow outlet permitting solvent delivery into and out of the column packed with SCX resin; and a pressure vessel solvent delivery system.

Materials Used. PolySULFOETHYL Aspartamide strong cation exchange resin (12 μm particle size; 300 angstrom pore size), hereafter referred to as SCX resin, is a product from The Nest Group, Inc. (Southborough, Mass.). Fused silica capillary tubing (200 μm inner diameter by 360 μm outer diameter; 320 μm inner diameter by 425 μm outer diameter) is a product of Polymicro Technologies (Phoenix, Ariz.). Fused silica capillary tubing fingertight fittings (product F-125), sleeves (products F-185X and F-186X for use with 360 μm outer diameter and 425 μm outer diameter tubing, respectively), ZDV unions (product P-720), and inline microfilters composed of a filter end fitting and microfilter union (product M-520) are products of Upchurch Scientific (Oak Harbor, Wash.).

1) SCX Resin Pre-Conditioning.

One-gram of SCX resin was transferred to a 50 mL centrifuge tube. Methanol (40 mL) was added and the slurry was vortexed vigorously for 10 min. The resin was pelleted by centrifugation (1000×g) and the supernatant decanted. A 1:1 solution of methanol:water (40 mL) was added and the slurry was vortexed vigorously for 10 min. The resin was again pelleted by centrifugation (1000×g) and the supernatant decanted. Water (40 mL) was added and the slurry was vortexed vigorously for 10 min. The resin was pelleted by centrifugation (1000×g) once more and the supernatant decanted. A solution of 0.2 M sodium phosphate (monobasic) and 0.3 M sodium acetate (40 mL) was added and the slurry was subjected to continuous mixing by rotation of the centrifuge tube for 24 hr. The resin was collected by centrifugation (1000×g) and the supernatant decanted. A solution of 2 M potassium chloride, 10 mM potassium phosphate (pH 3; 40 mL) was added and the slurry was subjected to continuous mixing by rotation of the centrifuge tube for 4 hr. The resin was collected by centrifugation (1000×g) and the supernatant decanted. A solution of 10 mM potassium phosphate (pH 3; 40 mL) was added and the slurry was subjected to continuous mixing by rotation of the centrifuge tube for 24 hr. The resin was collected by centrifugation (1000×g) and the supernatant decanted. Water (40 mL) was added and the slurry was subjected to continuous mixing by rotation of the centrifuge tube for 3 hr. The resin was collected by centrifugation (1000×g) and the supernatant decanted.

This latter process of washing the resin with water was repeated two more times. The final pre-conditioned resin pellet was stored at 4° C. prior to use.

2. Column Fabrication.

2.1. Columns Prepared Using Commercially Available Fused Silica Tubing Fittings (Refer to FIG. 1).

The method described was the same whether using either 200 μm inner diameter by 360 μm outer diameter or 320 μm inner diameter by 425 μm outer diameter fused silica capillary tubing. A 5 cm length of fused silica (1) was cut using a fused silica cutter. One end of the cut tubing was placed into a 1.5 cm length of capillary sleeve (2), such that the ends of the tubing and the sleeve were flush. The sleeve-covered end of the tubing was inserted into a fingertight fitting (3) such that the flush sleeve-covered end of the tubing protrudes through the fingertight fitting by approximately 1 mm. The fingei-tight fitting was then screwed into a filter end fitting (4), which was subsequently screwed into the filter end fitting opening (5) of a microfilter union (6). This furnished the completed column, which was then packed with SCX resin as described below (Section 3).

2.2. Columns Prepared with Embedded SCX Resin/Sol-Gel Frits (Refer to FIG. 2).

The method described was the same whether using either 200 μm inner diameter by 360 μm outer diameter or 320 μm inner diameter by 425 μm outer diameter fused silica capillary tubing. A sufficient amount of fused silica tubing (e.g. 1-2 m) was rinsed with methylene chloride (approximately 2 mL/m of tubing) and then purged and dried with a stream of nitrogen gas at 40 psi for 1 hr. The amount of tubing ultimately treated in this manner was determined by the desired number of columns to be fabricated. The initial length of dried tubing was then cut into 5 cm lengths using a fused silica cutter.

A previously reported method based on sol-gel chemistry (Zhang, X. and Huang, S. (2001) J. Chromatogr. A 910, 13-18) was modified extensively to produce columns with embedded frits. In the preferred method, a sol-gel solution was prepared by the sequential addition of trifluoroacetic acid (100 μL), methyltriethoxysilane (75 μL), water (10 μL) and methanol (200 μL) to a glass vial. The vial was sealed and the solution sonicated for 10 min. A 100 μl aliquot of this solution was added to 50 mg of SCX resin in a small vial containing a magnetic stir bar and the slurry was vortexed vigorously for 15 s. The vial was placed on top of a stir plate and the resin/sol-gel slurry was stirred continuously while one end of the individual 5 cm lengths of fused silica tubing (7) was dipped briefly (<1 s) into it. After dipping, the tubing was supported vertically, with the dipped end at the bottom, and allowed to rest for 24 h at 20-22° C. The tubing was then placed into a 90° C. oven for 4 hr. These latter two processes resulted in polymerization of the sol-gel solution and solidification of the resin/sol-gel mixture into an embedded frit at the end of the fused silica column (8). The tubing was then flushed with water (approximately 50 μL) in the direction from the embedded frit end to the open end of the tubing by use of a water-filled syringe and a fused silica tubing/syringe adapter. Residual water was purged from the tubing by forcing air through the tubing in the same direction as described above. The frit-embedded tubing was dried at 90° C. for 10 min. This furnished the completed column, which was then packed with SCX resin as described below (Section 3).

3. Reactor Packing

For each column to be packed, a mark was placed on the fused silica tubing 2 cm from the end housed within the microfilter union (for columns prepared as described in section 2.1) or that contained the embedded frit (for columns prepared as described in section 2.2). A fused silica tubing/syringe adapter was fitted onto the open end of the column. A slurry of pre-conditioned SCX resin in water (approximately 10 mg/mL) was prepared and drawn into a syringe. The syringe was fastened to the adapter and the slurry introduced into the column by depressing the syringe plunger with constant pressure. As the syringe plunger was depressed, the column was observed closely to monitor the progression of resin packing, which was readily visible through the fused silica tubing. When the height of the packed resin (9; FIGS. 1, 2) reached the 2 cm mark on the tubing, the pressure on the syringe plunger was stopped. The column was disassembled from the adapter, furnishing the completed SCX resin-packed column.

4. Flow Inlet and Flow Outlet Connections (Refer to FIGS. 1, 2).

The preparation of the flow inlet of the reactor was the same using either column format described in sections 2.1 and 2.2. The flow inlet of the reactor was prepared by inserting the open (top) end of the column tubing into a 1.5 cm length of capillary sleeve (10), such that the ends of the tubing and the sleeve were flush. The sleeve-covered end of the tubing was inserted into a fingertight fitting (11), such that the end of the flush sleeve-covered tubing protruded through the fingertight fitting by approximately 1 mm. The fingertight fitting was then screwed into one opening (12) of a ZDV union (13). The other opening of the ZDV union (14) served to receive the fingertight fitting (15) that housed the fused silica tubing forming the flow outlet (16) of the pressure vessel solvent delivery system (section 5).

For reactor devices constructed using columns prepared using fused silica tubing fittings (section 2.1), the flow outlet of the reactor device was prepared with additional components. One end of a 4 cm length of fused silica tubing (17; 200 μm inner diameter by 360 μm outer diameter) was inserted into a capillary sleeve (18; 1.5 cm in length) such that the ends of the tubing and the sleeve were flush. The sleeve-covered end of the tubing was inserted into a fingertight fitting (19) such that the end of the flush sleeve-covered tubing protruded through the fingertight fitting by approximately 1 mm. The fingertight fitting was then screwed into the fingertight fitting opening (20) of the microfilter union (6) that housed the column. In this format, the open end of the 4 cm length of tubing formed the flow outlet (21).

For reactor devices constructed using columns prepared with embedded SCX resin/sol-gel frits, no additional components were required to form the flow outlet of the reactor device. The flow outlet of the reactor was itself the frit embedded end of the packed column (21).

B. Apparatus and General Operation

For delivery to the reactor, a vial containing the sample or buffer was placed in the cavity (8b) in the lower block of the pressure vessel solvent delivery system. The latter was connected to a high-pressure gas cylinder, controlled by a low-pressure regulator. A valve (7) placed in-line between the gas cylinder and the pressure vessel allowed for simple pressure-on/off operation. A bleed valve (6b) on a split line was incorporated to allow for de-pressurization of the pressure vessel when needed (See FIG. 3).

In one embodiment, the pressure vessel is capable of housing a single vial. In another embodiment, the pressure vessel is capable of housing multiple vials and thus able to run multiple samples simultaneously (see FIG. 4). In another embodiment, the pressure vessel is capable of housing multiple vials with the ability to individually control pressure applied to each sample. More detailed operating instructions are been described above (see FIG. 5).

To change the solution being delivered to the device, the pressure vessel first must be de-pressurized. This was achieved by halting the pressure using the on/off valve, and bleeding the high-pressure gas from within the pressure vessel using the bleed valve. Once de-pressurized, the cavity within the pressure vessel became accessible and the vial containing the subsequent solution to be delivered could be substituted.

Two primary methods of solution delivery to the device were used in the methods described herein. In a first method, solutions were applied to the device continuously, to the point of depletion, if necessary. If the solution was depleted, the pressure vessel delivered a stream of gas to the device, effectively de-solvating the SCX resin to dryness. Most transitions between different solutions occurred with the device in this dehydrated state. Second, solutions were “infused” into the device by immediately halting delivery once the SCX resin was completely saturated in the solution and the solution just begins to emerge from the flow outlet. The device was incubated in this saturated state to allow for chemical and/or enzymatic reactions to proceed. The infusion process typically consumed less than 2 μL of solution.

In a second method, solution delivery could be achieved by connecting the device in-line to a High Performance Liquid Chromatography (HPLC) or Fast Performance Liquid Chromatography (FPLC) pump. The required solutions could be manually injected through an injector port, or automatically injected with an autosampler. Alternatively, solution delivery could be achieved using a syringe connected to the device.

C. Detailed Method of Operation

Buffers and Reagents

All buffers where indicated were filtered through a 0.2 μm syringe filter prior to use to prevent any particulate contaminants from entering the Reactor. All buffers containing trypsin were prepared immediately prior to use. All solutions containing NH₄HCO₃ were freshly prepared.

The following buffers and reagents were prepared as required:

| | |
|---|---|
| X: | 50 mM H₃PO₄ (filtered) |
| W: | MilliQ-H₂O (filtered) |
| W18: | H₂¹⁸O |
| K1: | 10 mM KH₂PO₄ pH 3.0 (filtered) |
| K2: | 10 mM H₃PO₄, prepared using W18 (filtered) |
| A1: | 1 M NH₄HCO₃ |
| A2: | 100 mM NH₄HCO₃ |
| A3: | 1 M NH₄HCO₃, prepared using W18 |
| A4: | 100 mM NH₄HCO₃, prepared using W18 |
| T1: | 200 mM Tris-HCl, pH 8.0 |
| T2: | 200 mM Tris-HCl, pH 8.0, prepared using W18 |
| R1: | 1 M dithiothreitol (DTT) |
| R2: | 0.5 μL R1 + 0.5 μL A2 + μL W |
| | (100 mM DTT, 10 mM NH₄HCO₃) |
| R3: | 1 M DTT, prepared using W18 |
| R4: | 0.5 μL R3 + 0.5 μL + A4 + 4 μL W18 |
| | (100 mM DTT, 10 mM NH₄HCO₃ in H₂¹⁸O) |
| C1: | 100 mM iodoacetamide |
| C2: | 100 mM iodoacetamide, prepared using W18 |
| D1: | 1.25 μL TS + 0.5 μL A1 + 3.25 μL W |
| | (0.5 mg/mL trypsin, 100 mM NH₄HCO₃) |
| D2: | 2.5 μL T1 + 2.5 μL W |
| | (100 mM Tris-HCl pH 8.0) |
| D3: | 1.25 μL TS + 0.5 μL A1 + 0.5 μL C1 + 2.75 μL W |
| | (0.5 mg/mL trypsin, 100 mM NH₄HCO₃, |
| | 10 mM iodoacetamide) |
| D4: | 2.5 μL T1 + 0.5 μL C1 + 2 μL W |
| | (100 mM Tris-HCl pH 8.0, 10 mM iodoacetamide) |
| D5: | 1.25 μL TS18 + 0.5 μL A3 + 0.5 μL C2 + 2.75 μL W18 |
| | (0.5 mg/mL trypsin, 100 mM NH₄HCO₃, |
| | 10 mM iodoacetamide in H₂¹⁸O) |
| D6: | 2.5 μL T2 + 0.5 μL C2 + 2 μL W18 |
| | (100 mM Tris-HCl pH 8.0, 10 mM iodoacetamide in H₂¹⁸O) |
| TS: | 2 mg/mL trypsin |
| TS18: | 2 mg/mL trypsin, prepared using W18 |

Procedure

The methods outlined below encompass several functions of the apparatus, including enzymatic digestion, chemical modification and isotopic labeling of polypeptides. The Basic procedure describes the enzymatic digestion of polypeptides by trypsin. As illustrated in the Alternate steps, the basic protocol can be amended to accommodate additional and/or modified reactions to enable chemical modification or labeling, for example. These examples showcase the flexibility of the methodology, and in no way define the entire scope of potential applications. Alternate A describes a method that uses a different digestion buffer and second method of introducing enzyme to the sample. Alternate B augments the basic digestion protocol with the reduction of disulfide bonds between cysteine residues and subsequent alkylation of the free sulfhydryls. Alternate C details the isotopic labeling of tryptic peptides at their C-termini with ¹⁸O. The Alternate methods can be used concurrently with the basic protocol to achieve the desired functionality.

Basic Protocol (Enzymatic Digestion of Protein Substrates)

1. Device Equilibration. Devices were equilibrated briefly with K1 prior to loading samples.

2. Sample Preparation. Protein samples were acidified by diluting with X. Samples were then centrifuged for 10 minutes at 4° C. at 20800×g to pellet any insoluble material. The supernatants were transferred to fresh vials and placed in the pressure vessel for delivery to the device.

3. Sample Loading. Samples were loaded onto the device. Solution exiting the device flow outlet was collected for further analysis to determine the extent of unbound protein. Sample was loaded to the point of depletion and the SCX resin was rendered dry.

4. First Wash. Residual contaminants were washed from the device using 30 μL of K1.

5. Second Wash. The device was washed with 15 μL of W1.

6. Enzymatic Digestion. To enzymatically digest immobilized proteins, the device was infused with D1.

7. Device Incubation. To allow enzymatic digestion to occur, the device hydrated in D1 was incubated at room temperature for 1 hour.

8. Elution. Products were eluted from the device using 12-20 μL of A2 and collected in a vial.

9. Storage. Products were stored at −80° C.

Alternate A: Enzymatic Digestion of Protein Substrates

An alternative method for enzymatic digestion changes the point at which enzyme is introduced. With reference to the basic protocol described above, instead of applying trypsin to the proteins immobilized to the device, trypsin could be introduced concomitant with the sample during the sample loading step. Trypsin activity is inhibited under these conditions due to the acidic nature of X. Likewise, any proteinaceous enzyme could be applied to the device simultaneously with its targeted substrate, since conditions favoring substrate immobilization to the device should also promote enzyme immobilization. Referring to the Basic protocol again, the point where trypsin is usually introduced can be replaced with simply the introduction of buffer conditions that would allow trypsin activity to ensue.

1. Device Equilibration. Devices were equilibrated briefly with K1 prior to loading samples.

2. Sample Preparation. Protein samples were acidified by diluting with X. Samples were then centrifuged for 10 minutes at 4° C. at 20800×g to pellet any insoluble material. The supernatants were transferred to fresh vials. To each sample, 0.5 μL of TS was added. Samples were placed in the pressure vessel for delivery to the Reactor.

3. Sample Loading. Samples were loaded onto the device. Solution exiting the device flow outlet was collected for further analysis to determine the extent of unbound protein. Sample was loaded to the point of depletion and the SCX resin was rendered dry.

4. First Wash. Residual contaminants were washed from the device using 30 μL of K1.

5. Second Wash. The device was washed with 15 μL of W1.

6. Enzymatic Digestion. To enzymatically digest immobilized proteins, the device was infused with D2, thereby activating trypsin through the favourable pH of the buffer.

7. Device Incubation. To allow the enzymatic digestion to occur, the device hydrated in D2 was incubated at room temperature for 1 hour.

8. Elution. Products were eluted from the device using 12-20 μL of A2 and collected in a vial.

9. Storage. Products were stored at −80° C.

Alternate B: Serial Chemical and Enzymatic Transformation of Protein Substrates

1. Equilibration. Devices were equilibrated briefly with K1 prior to loading samples.

2. Sample Preparation. Protein samples were acidified by diluting with X. Samples were then centrifuged for 10 minutes at 4° C. at 20800×g to pellet any insoluble material. The supernatants were transferred to fresh vials and placed in the pressure vessel for delivery to the Reactor.

3. Sample Loading. Samples were loaded onto the device. Solution exiting the device flow outlet was collected for further analysis to determine the extent of unbound protein. Sample was loaded to the point of depletion and the SCX resin was rendered dry.

4. First Wash. Residual contaminants were washed from the device using 30 μL of K1.

5. Second Wash. The device was washed with 15 μL of W1.

6. Chemical Transformation. To convert disulfide-bonded cysteine residues into their sulfhydryl form, the reducing agent, DTT, was applied to the immobilized proteins by infusing R2 into the device.

7. Device Incubation. The device, hydrated in R2, was incubated at room temperature for 30 minutes.

8. Third Wash. After the incubation period, R2 was expelled from the device and the SCX resin was rendered dry. The device was then washed with 2 μL of K1 to remove residual R2.

9. Enzymatic and Chemical Transformation. To prevent disulfide bonds from reforming, the sulfhydryl groups of cysteine residues were covalently modified by reaction with iodoacetamide. Simultaneously, proteins were enzymatically digested with trypsin. This was accomplished by infusing the device with D3. To combine Alternate A and Alternate B, the device should be infused with D4.

10. Device Incubation. To allow the enzymatic and chemical transformations to occur, the device hydrated with the buffer in step 9 was incubated at room temperature for 1 hour.

11. Elution. Products were eluted from the device using 12-20 μL of A2 and collected in a vial.

12. Storage. Products were stored at −80° C.

Alternate C: Serial Chemical and Enzymatic Transformation with Isotopic Labeling of Products

1. Equilibration. Devices were equilibrated briefly with K1 prior to loading samples.

2. Sample Preparation. Protein samples were acidified by diluting with X. Samples were then centrifuged for 10 minutes at 4° C. at 20800×g to pellet any insoluble material. The supernatants were transferred to fresh vials and placed in the pressure vessel for delivery to the Reactor.

3. Sample Loading. Samples were loaded onto the device. Solution exiting the device flow outlet was collected for further analysis to determine the extent of unbound protein. Sample was loaded to the point of depletion and the SCX resin was rendered dry.

4. First Wash. Residual contaminants were washed from the device using 30 μL of K1.

5. Second Wash. The device was washed with 15 μL of W18.

6. Chemical Transformation. To convert disulfide-bonded cysteine residues into their sulfhydryl form, the reducing agent, DTT, was applied to the immobilized proteins by infusing R4 into the device.

7. Reactor Incubation. The device, hydrated in R4, was incubated at room temperature for 30 minutes.

8. Third Wash. After the incubation period, R4 was expelled from the device and the device was run dry. The device was then washed with 2 μL of K2 to remove residual R4.

9. Enzymatic and Chemical Transformation. To prevent disulfide bonds from reforming, the sulfhydryl groups of cysteine residues were covalently modified by reaction with iodoacetamide. Simultaneously, proteins were enzymatically digested with trypsin and the products isotopically labeled at their C-termini with ¹⁸O. This was accomplished by infusing the device with D5. To combine Alternate A and Alternate C, the device should be infused with D6.

10. Device Incubation. To allow the enzymatic and chemical transformations to occur, the device hydrated with the buffer from step 9 was incubated at room temperature for 1 hour.

11. Elution. Products were eluted from the device using 12-20 μL of A4 and collected in a vial.

12. Storage. Products were stored at −80° C.

### EXAMPLE 1

### Tryptic Digestion of Standard Proteins Using the Reactor

A mixture of four commercially available proteins (rabbit glycogen phosphorylase B, bovine serum albumin, bovine carbonic anhydrase, and horse heart myoglobin) was prepared in 50 mM phosphoric acid. Each protein was prepared to give a final concentration of 1 pmol/μL in the mixture. 10 μL of this mixture (10 pmol each protein) was applied to an SCX column, washed, and digested according to the instructions outlined in the Basic Protocol of section C, above. The resulting elution fraction (˜15 μl) was diluted 5-fold and 2 μL of the diluted fraction was analyzed on a LC-QSTAR mass spectrometer. Assuming complete digestion and recovery of peptides, this amounted to 267 fmol of each digested protein analyzed by LC/MS.

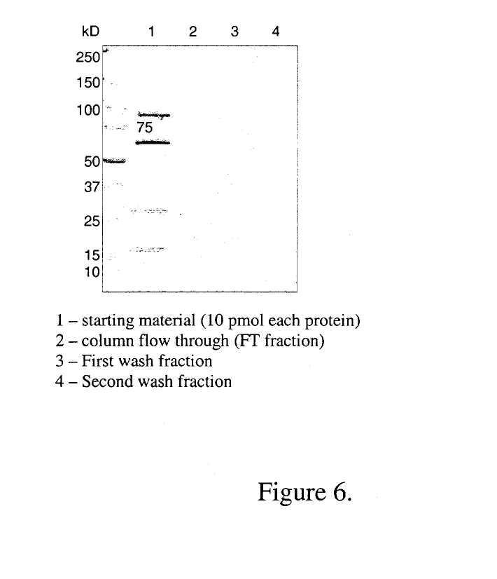

FIG. 6 illustrates that the entire protein sample applied to the column was immobilized onto the SCX resin. If immobilization did not occur or if proteins were displaced from the SCX resin during the subsequent wash steps, proteins would elute in the various wash fractions and would be detected on the gel.

The resulting MS data were queried against protein databases and each of the 4 proteins was unambiguously identified. Using the Mascot searching algorithm (Matrix Science), the entire NCBI database was searched, producing the following results (top 20 hits shown):

| | |
|---|---|
| gi\|6093713 | GLYCOGEN PHOSPHORYLASE, MUSCLE FORM |
| | (MYOPHOSPHORYLASE) |
| gi\|223003 | phosphorylase b, glycogen [Oryctolagus cuniculus] |
| gi\|14916625 | GLYCOGEN PHOSPHORYLASE, MUSCLE FORM |
| | (MYOPHOSPHORYLASE) |
| gi\|225897 | glycogen phosphorylase [Homo sapiens] |
| gi\|2190337 | (X58989) serum albumin [Bos taurus] |
| gi\|2506462 | MYOGLOBIN |
| gi\|115453 | CARBONIC ANHYDRASE II |
| | (CARBONATE DEHYDRATASE II) (CA-II) |
| gi\|164318 | (M36787) albumin [Sus scrofa] |
| gi\|115460 | CARBONIC ANHYDRASE II |
| | (CARBONATE DEHYDRATASE II) (CA-II) |
| gi\|13124699 | SERUM ALBUMIN PRECURSOR |
| | (ALLERGEN CAN F 3) |
| gi\|1351908 | SERUM ALBUMIN PRECURSOR |
| | (ALLERGEN FEL D 2) |
| gi\|127691 | MYOGLOBIN |
| gi\|127641 | MYOGLOBIN |
| gi\|4506351 | phosphorylase, glycogen; brain [Homo sapiens] |
| gi\|87573 | glycogen phosphorylase (EC 2.4.1.1) - human (fragment) |
| gi\|127685 | MYOGLOBIN |
| gi\|127634 | MYOGLOBIN |
| gi\|70559 | myoglobin - Indian elephant |
| gi\|11935049 | (AF304164) keratin 1 [Homo sapiens] |
| gi\|127664 | MYOGLOBIN |

The Mascot search algorithm also lists the unique peptides found for each protein. Some peptides are listed more than once, corresponding to the fragmentation of the same peptide at different charge states (+2 or +3). This further reinforces the certainty of the presence of the particular peptide.

Number of Peptides Identified:

Phosphorylase B—26 peptides

BSA—13 peptides

Myoglobin—10 peptides

Carbonic anhydrase—10 peptides

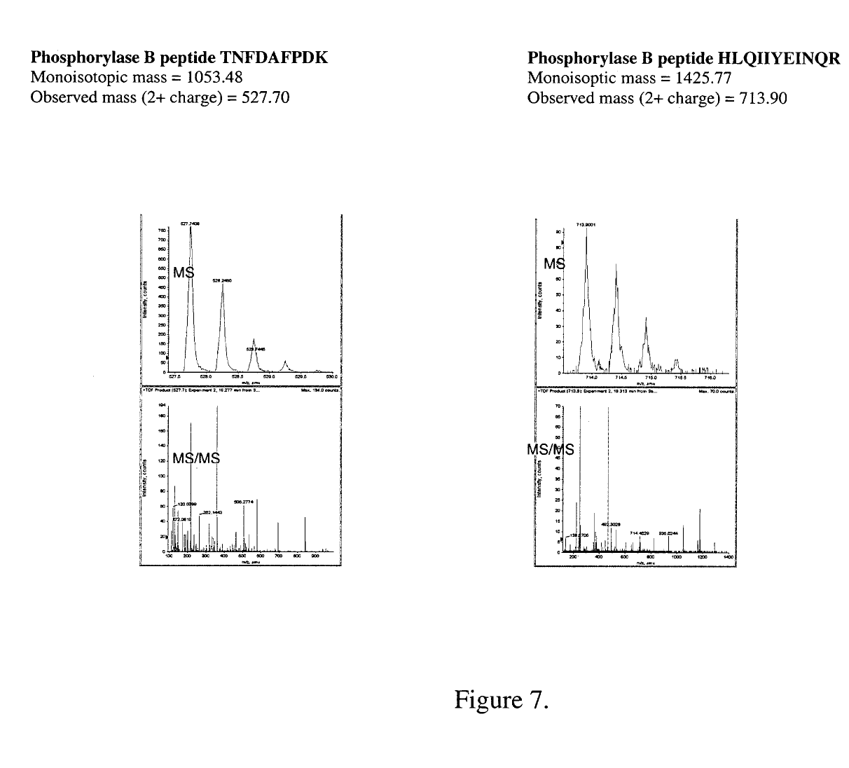

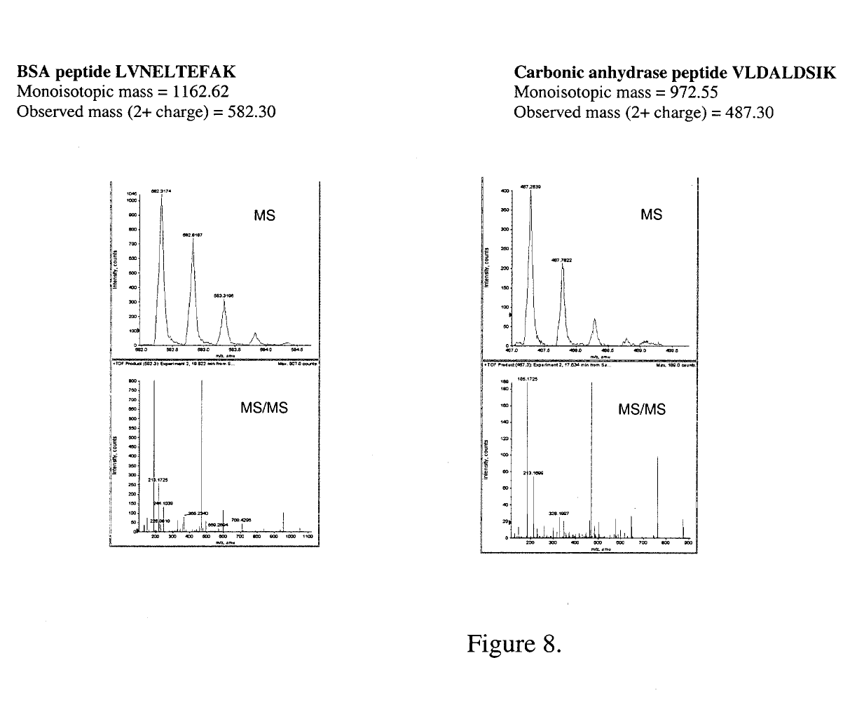

Representative MS and MS/MS spectra acquired for this analysis are presented in FIGS. 7 and 8.

Collectively, these results demonstrate that the standard proteins were concentrated and immobilized onto the SCX resin and were digested very efficiently by trypsin using the reactor methodology. The resulting peptides were isolated and analyzed by MS without further purification, and database searching of the MS derived data unequivocally correctly identified each of the 4 proteins.

### EXAMPLE 2

### Tryptic Digestion of Immunopurified Human Proteins Using the Reactor

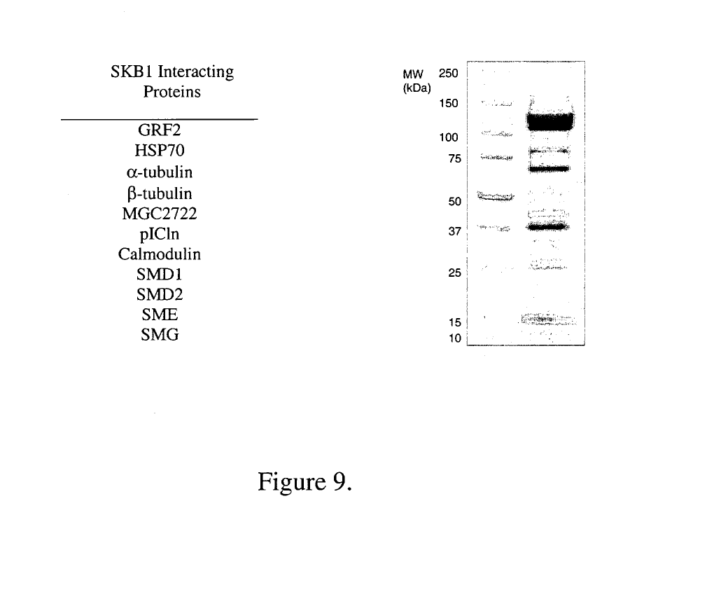

Standard protocols were employed to immunopurify an epitope-tagged protein (bait) from cultured human cells (hEK293 cells) and proteins that complex or interact with the bait protein. Briefly, human cells were transfected with DNA encoding the bait protein. Cells were cultured for 2 days and then were harvested and lysed using detergent-containing buffer. The clarified lysate was subjected to immunopurification using an immobilized antibody resin against the specific epitope. The resin was collected, and the immunopurified bait and associated proteins were eluted from the antibody resin using 50 mM phosphoric acid. A portion of the eluent (containing ˜5 μg total protein) was analyzed by SDS-PAGE (see FIG. 9) to characterize the immunopurified complex and to ensure that the immunopurification process was successful. Another portion of the eluent (˜500 ng total protein, or ˜10 pmol total protein) was subjected to trypsin digestion using the method of Alternate B, above. The elution fraction collected from the device following digestion was analyzed using an LCQ-DECA mass spectrometer.

The bait protein exemplified in this case, human SKB 1, has been previously shown to interact with several specific associating proteins. Each of these associating proteins, a list of which is shown in FIG. 9, was identified using this method.

These results exemplify the utility and advantages of the reactor based methodology. The protein sample in this case was isolated from human cells. While the immunopurification process does purify the protein complex, the sample is nonetheless contaminated with other cellular components and reagents used during the process. In particular, residual detergent from the lysis procedure is extremely problematic to MS analysis. The reactor-based method circumvents these problems. The amount of protein sample required for the reactor methodology is appreciably less than that required for standard gel-based methods (the data from the reactor-based experiment was obtained using approximately 10% of the amount of material needed for the gel-based experiment). Furthermore, in the case of the latter, each protein band from the gel (e.g. FIG. 9) must be excised, digested and analyzed separately and only those bands visible on the gel would be processed. The reactor-based method streamlines the entire process into a single, integrated system.

### EXAMPLE 3

### Reduction and Alkylation of Sulfhydryl Groups of Peptides

TABLE 1

| | | | Disulfide- | Singly- | Doubly- |
|---|---|---|---|---|---|
| | | Reduced | bonded | Alkylated | Alkylated |
| Peptide | Sequence | MW | MW | MW | MW |
| 1 | CYFQNCPK | 1001.41 | 999.39 | 1058.47 | 1115.53 |
| 2 | CYFQNCPR | 1029.42 | 1027.40 | 1086.48 | 1143.54 |
| 3 | CDPGYIGSR | 966.42 | 1931.84 | 1023.48 | — |

A mixture of 3 cysteine-containing peptides in their disulfide-bonded (oxidized) state, listed in Table 1, above, was prepared in 50 mM H₃PO₄, and 50 pmol total peptide was loaded onto a device. The immobilized peptides were then processed according to the instructions in Section C, Alternate B, above.

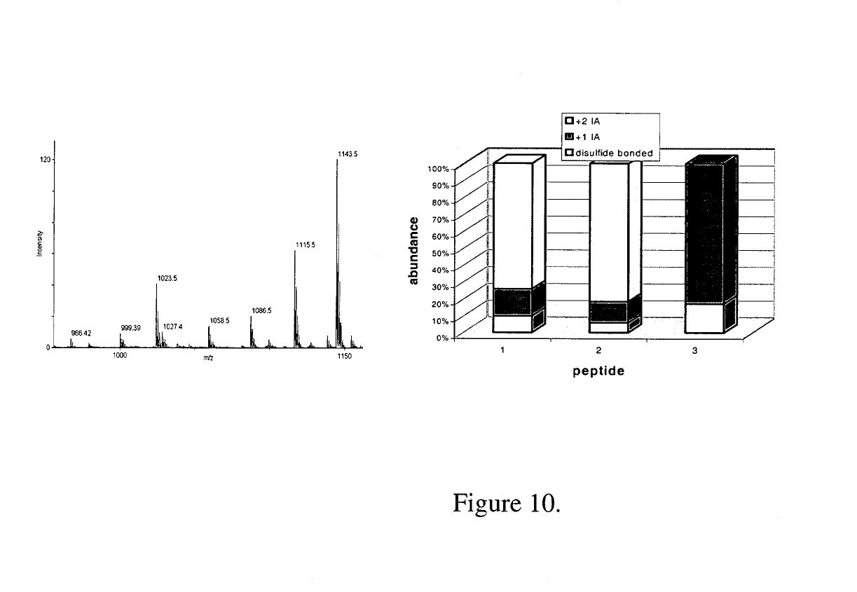

Shown in FIG. 10 is the MALDI-Tof mass spectrum of the peptide mixture following chemical reduction and alkylation using the reactor based method. Comparing relative peak intensities, it can be seen that, for each peptide, more than 80% are fully alkylated, as illustrated graphically. These results clearly demonstrate the feasibility of chemical transformation of immobilized polypeptides.

### EXAMPLE 4

### Serial Transformation: Reduction, Alkylation and Tryptic Digestion of Proteins.

A mixture of 4 commercially available proteins (rabbit glycogen phosphorylase B, bovine serum albumin, chicken ovalbumin, horse heart myoglobin) was prepared in 50 mM phosphoric acid, each at a concentration of 2 pmol/μL. 10 μL of this mixture (20 pmol of each protein) was loaded onto a reactor. The immobilized proteins were then processed according to the instructions in Section C, Alternate B, above.

A parallel sample, which served as a control, was processed according to the instructions outlined in Section C, Basic Protocol, above. Thus, the first sample was subjected to chemical reduction, alkylation by iodoacetamide and enzymatic digestion with trypsin. In contrast, the control sample was simply digested with trypsin.

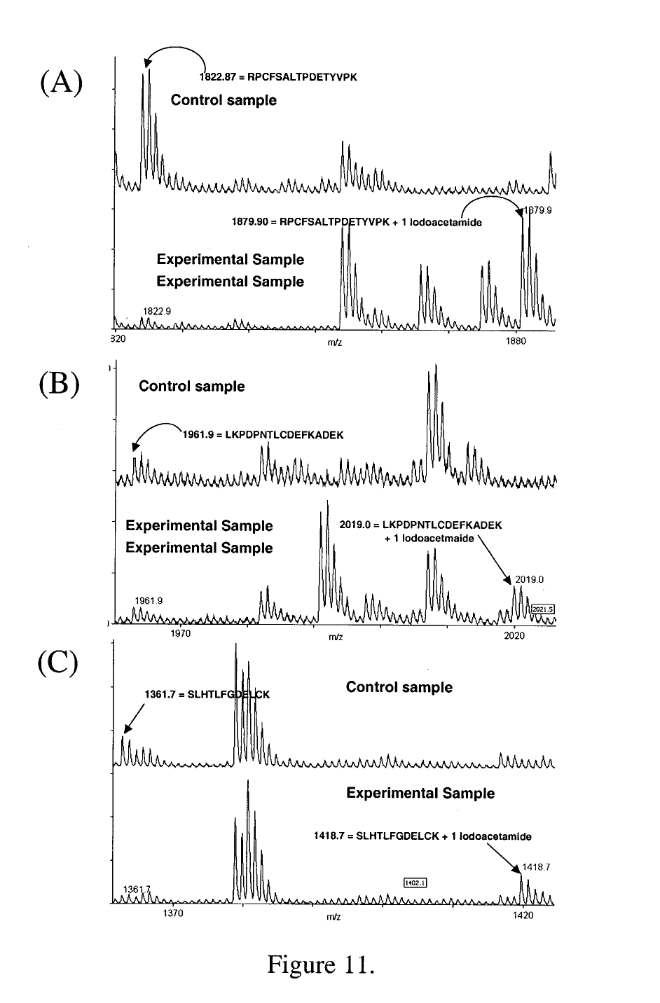

Bovine Serum Albumin (BSA) contains 17 disulfide bonds making it an ideal substrate for this experiment. The MALDI-Tof peptide mass fingerprints from both the control and experimental samples were searched using Mascot and the identified BSA peptides were compared for the presence of iodoacetamide-modified cysteine residues. The representative spectra shown in FIG. 11 illustrate the modification of cysteine residues for three tryptic peptides from BSA.

Other cysteine-containing BSA tryptic peptides that were alkylated and identified in the experimental sample, but not in the control sample included:

MPCTEDYLSLILNR+1 IA

YNGVFQECCQAEDK+2 IA

NECFLSHKDDSPDLPK+1 IA

CCAADDKEACFAVEGPK+3 IA

ECCHGDLLECADDRADLAK+3 IA

CCTKPESERMPCTEDYLSLILNR+1 IA

TCVADESHAGCEKSLHTLFGDELCK+2 IA

Similarly, cysteine-containing tryptic peptides from Phosphorylase B and Ovalbumin were identified as being modified by iodoacetamide. These results clearly demonstrate that both chemical and enzymatic transformation of proteins are efficiently achieved using the reactor-based methodology.

### EXAMPLE 5

### Protein Quantitation Using Stable Isotope Labeling with H₂¹⁸O

Two separate samples (A and B), each containing the same eight commercially available proteins but at different absolute amounts, were prepared in 50 mM H₃PO₄ (100 μL total volume) as outlined in Table 2, below. In Sample A, the proteins ranged in absolute amounts from 100 fmol (Cytochrome C) to 40 pmol (Myoglobin). In the case of Sample B, the proteins ranged in absolute amounts from 200 fmol (Carbonic Anhydrase) to 30 pmol (Ovalbumin). The ratio of the individual proteins present in Sample A to Sample B varied from 0.05 (or 1:20, Aldolase) to 10 (or 10:1, Myoglobin). The total protein content in both samples was approximately 3.6 μg. Thus, the two samples represent complex mixtures of proteins present in a broad range of absolute amounts and, therefore, were representative of complex mixtures that would be obtained from real biological samples. Furthermore, the total protein content of each sample was also representative of the amount of protein material that would realistically be isolated from a biological sample. Therefore, as designed, Sample A and Sample B represented a worthy model system for interrogation by the practice of stable isotope labeling using ¹⁸O-labeled water (H₂¹⁸O) for quantitative proteomics analysis.

TABLE 2

| | | | | Experimental |
|---|---|---|---|---|
| | Absolute Amount of | | | Protein Ratio |
| | Protein in | | Theoretical | Determined By |
| | Each Sample (pmol) | | Protein Ratio | MS Analysis |
| Protein | Sample A | Sample B | A:B | A:B |
| Aldolase | 0.2 | 4 | 0.05 | 0.08 |
| Albumin | 2 | 20 | 0.1 | 0.15 |
| Carbonic | 0.5 | 0.2 | 2.5 | 1.63 |
| Anhydrase | | | | |
| Cytochrome C | 0.1 | 0.3 | 0.33 | 0.44 |
| Enolase | 10 | 10 | 1 | 0.95 |
| Myoglobin | 40 | 4 | 10 | 8.70 |
| Ovalbumin | 30 | 30 | 1 | 0.89 |
| Phosphorylase B | 10 | 2 | 5 | 6.00 |

Sample A and Sample B were each separately subjected to sample processing with reactors fabricated using columns prepared with embedded SCX resin/sol-gel frits.

Sample A was selected for processing so that the peptides would be labeled at their carboxy terminus with ¹⁸O. Thus, Sample A was applied to the device, washed, treated with DTT, digested with trypsin and alkylated with iodoacetamide according to the instructions outlined above (Method Alternate C; using D6 at step 9) using the appropriate H₂¹⁸O-enriched buffer reagents. The resulting elution fraction (18 μL) was collected and 1.5 μL of 50% formic acid (in H₂¹⁸O) was added generating the Sample A analyte.

Sample B was selected for standard sample processing. Thus, Sample B was applied to a second device, washed, treated with DTT, digested with trypsin and alkylated with iodoacetamide according to the instructions outlined above (Method Alternate B; using D4 at step 9) using the appropriate buffer reagents that were prepared in natural abundance water (H₂¹⁶O). The resulting elution fraction (18 μL) was collected and 1.5 μL of 50% formic acid (in H₂¹⁶O) was added generating the Sample B analyte.

A portion of the Sample A analyte (2 μL) was analyzed without further purification by LC/MS/MS using an Agilent 1100 HPLC coupled to an AB/Sciex QStar mass spectrometer. The resulting MS data were queried against a database containing the sequences of the eight proteins using the Mascot searching algorithm. The searching parameters were set to include a variable modification for ¹⁸O labeling of the carboxy terminus of the peptide. The search result report generated indicated that the proteins were correctly identified. The Mascot result report also listed the unique peptides identified for each protein, which indicated that the greater majority of peptides identified by MS sequencing, were labeled at their carboxy terminus with ¹⁸O.

In a separate experiment, equal portions of the Sample A analyte and the Sample B analyte were mixed, and 2.5 μL of the resultant 1:1 mixture was analyzed by LC/MS/MS without further purification using an Agilent 1100 HPLC coupled to an AB/Sciex QStar mass spectrometer. The resulting MS data were queried against a database containing the sequences of the eight proteins using the Mascot searching algorithm. The searching parameters were set to include a variable modification for ¹⁸O labeling of the carboxy terminus of the peptide. The search result report generated indicated that the proteins were correctly identified.

For the experiment described above for the 1:1 mixture, tryptic peptides generated from Sample A are labeled at their carboxy terminus with the heavy version of oxygen (i.e. ¹⁸O) and tryptic peptides generated from Sample B are labeled at their carboxy terminus with the light version of oxygen (i.e. ¹⁶O). Thus, when mixed, the heavy- and light-labeled version of peptides will form an analyte pair of identical peptide sequence and chemical composition but of different mass, which will appear as separate mass peaks in a mass spectrum. Using known practices in the art [Stewart, I. I., Thomson, T. and Figeys, D. (2001) Rapid Commun. Mass Spectrom., 15, 2456-2465; Reynolds, K. J., Yao, X., and Fenselau, C. (2001) J. Proteome Res. 1, 27-33] the mass peaks are deconvoluted and an intensity assigned to the heavy and light mass peaks. The ratio between these intensities is equal to the ratio between the amount of light and heavy version of the peptide. By extension, this provides a ratio of the absolute amount of a given protein in Sample A to the absolute amount of the same protein in Sample B.

Several peptides that were identified above for each protein were selected for additional analysis. The mass spectrum for a given peptide-pair was retrieved, and intensities were assigned to the heavy and light versions using the practice described above. In each case, the intensity of the heavy-peptide from Sample A was divided by the intensity of the light-peptide from sample B. For each of the eight proteins, this provided an experimentally derived ratio for the absolute amount of the protein in Sample A to Sample B (Table 2). The experimentally derived ratios are in strong agreement with the theoretical protein ratio (Table 2).

These results demonstrate that sample processing using the reactor-based device and the procedures described herein provide accurate results on the relative amount of a given protein present in two samples. Therefore, the reactor-based device and procedures provide a reliable method to perform quantitative proteomics analysis.

Furthermore, these results demonstrate that the standard proteins were concentrated and immobilized onto the SCX resin, and were digested very efficiently by trypsin using the reactor methodology, even though there existed a difference in the complexity of the proteins in Sample A and Sample B.

In the case of Sample A, which was processed for purposes of ¹⁸O-labeling, the results indicate that a protein sample present in a medium containing only H₂¹⁶O can be transferred into a medium containing highly enriched H₂¹⁸O. Furthermore, this is accomplished without the need for drying down the sample and reconstituting it in highly enriched H₂¹⁸O or significantly diluting the sample into highly enriched H₂¹⁸O. If the efficient transfer did not occur, then the greater majority of peptides identified by MS sequencing would not have been labeled at their carboxy terminus with ¹⁸O.

Lastly, the results demonstrate that the efficient labeling of peptides with ¹⁸O can be achieved while using only minimal amounts of highly enriched H₂¹⁸O. By application of the method, less than 35 μL of H₂¹⁸O is consumed in the course of sample processing using the reactor device. Therefore, the method represents a very cost effective method for stable isotope labeling with ¹⁸O, and by extension, a cost effective method for performing quantitative proteomics analysis.

It will be appreciated that the methods and apparatus of the present invention can be incorporated in the form of a variety of embodiments, only a few of which are described herein. It will be apparent to those skilled in the art that other embodiments exist that do not depart from the spirit of the invention.

Further, whilst the examples disclosed herein use nanogram quantities of proteins, the reactor, device and apparatus may also be used on a larger scale to handle milligram or even gram quantities of material. Any modifications required to facilitate such scale-up (for example, to the solvent delivery system) will be apparent to those skilled in the art.

We claim:

1. A method for processing protein or peptide samples, comprising:
(i) reversibly immobilizing said peptide or protein samples onto a solid support;
(ii) transforming the immobilized peptide or protein by solid-phase chemical or enzymatic means, or a combination thereof;
(iii) eluting the resulting peptide- or protein-derived fragments from said solid support; and
(iv) recovering the fragments.

2. The method of claim 1, wherein the solid support is an ion-exchange resin.

3. The method of claim 2, wherein the resin is a cation exchange resin.

4. The method of claim 1, wherein the transformation is digestion of the peptide or protein.

5. The method of claim 4, wherein the digestion is carried out using an enzyme.

6. The method of claim 5, wherein the enzyme is trypsin.

7. The method of claim 2, where the immobilization and elution are pH-dependent.

8. The method of claim 2, where the immobilization and elution are ionic strength-dependent.

9. The method of claim 1, wherein the transformation is chemical transformation of the peptide or protein.

10. The method of claim 9, wherein the transformation is reduction of the peptide or protein.

11. The method of claim 9, wherein the transformation is alkylation of the peptide or protein.

12. The method of claim 11, wherein the transformation is alkylation of cysteine residues.

13. The method of claim 1, wherein the processing is carried out in semi-batch mode.

14. A method for the processing of peptide or protein samples according to the method of claim 1, wherein the total peptide or protein amount is less than about 1 pmol.

15. A method for the processing of peptide or protein samples according to the method of claim 14, wherein the total peptide or protein amount is less than about 100 fmol.

16. A method for the processing of peptide or protein samples according to the method of claim 15, wherein the total peptide or protein amount is less than about 10 fmol.

17. A method for the processing of peptide or protein samples according to the method of claim 16, wherein the total peptide or protein amount is less than about 1 fmol.

18. The method of claim 1, wherein said fragments are suitable for use in mass spectrometer analysis.

19. The method of claim 1, further comprising washing said samples after step (i) with one or more buffers before step (ii).

20. The method of claim 1, further comprising dehydrating said protein or peptide samples on said solid support by purging all solvents used to dissolve said samples after step (i), followed by rehydrating said protein or peptide samples before step (ii).

21. The method of claim 20, wherein said dehydrating and rehydrating steps are separated by a period of storage time.

22. The method of claim 1, wherein said solid support is ion-exchange resin packed in a column.

23. The method of claim 22, wherein said resin is strong cation exchange resin.

24. The method of claim 1, wherein said protein or peptide is immunoprecipitated from a biological sample.

25. The method of claim 24, wherein said biological sample is a lysate of a cell expressing said protein or peptide.

26. The method of claim 1, wherein at least one of steps (i), (iii), or (iv) is facilitated by a solvent delivery system.

27. The method of claim 26, wherein said solvent delivery system is capable of delivering solvents to more than one independently operating solid supports.

28. A method of determining the ratio of proteins in a first and a second samples, the method comprising:
(a) processing said first sample using the method of claim 5 in the presence of H₂¹⁸O, thereby labeling the carboxy termini of the peptide fragments resulting from the digestion with ¹⁸O;
(b) processing said second sample using the method of claim 5 at the presence of H₂¹⁶O, thereby labeling the carboxy termini of the peptide fragments resulting from the digestion with ¹⁶O;
(c) analyzing all peptide fragments of steps (a) and (b) using mass spectrometry by determining the ratio of each pair isotopically labeled fragment, thereby determining the ratio of proteins in said first and second sample.

29. An apparatus for processing protein or peptide samples according to claim 1, comprising:
(i) a reactor comprising a solid support;
(ii) a solvent delivery system; and
(iii) a means for connecting (i) and (ii).
